(19) United States
(12) Patent Application Publication
(10) Pub. No.: US 2025/0216314 A1
(43) Pub. Date: Jul. 3, 2025

(54) CELL ANALYSIS METHOD AND CELL ANALYSIS SYSTEM

(71) Applicant: RESEARCH & BUSINESS FOUNDATION SUNGKYUNKWAN UNIVERSITY, Suwon-si (KR)

(72) Inventors: Soo Yeon CHO, Suwon-si (KR); Young Ho SONG, Suwon-si (KR); Changyu TIAN, Suwon-si (KR)

(73) Assignee: RESEARCH & BUSINESS FOUNDATION SUNGKYUNKWAN UNIVERSITY, Suwon-si (KR)

(21) Appl. No.: 18/886,209

(22) Filed: Sep. 16, 2024

(30) Foreign Application Priority Data
Dec. 27, 2023 (KR) 10-2023-0193731

(51) Int. Cl. G01N 15/1429; G01N 15/10; G01N 15/14; G01N 15/1434; G16B 50/30

(52) U.S. Cl. CPC G01N 15/1429; G01N 15/1434; G16B 50/30; G01N 2015/1006; G01N 2015/1443; G01N 2015/1493; G01N 2015/1497

(57) ABSTRACT

A cell analysis method and a cell analysis system are provided. The cell analysis method includes: providing a flow unit to provide a path in which a cell flows, the flow unit including a light emitting layer to, in response to being irradiated by a first light in a first wavelength band, emit a second light in a second wavelength band; irradiating the light emitting layer with the first light; emitting the second light from the light emitting layer in response to the irradiation of the first light; acquiring an image formed by reacting the cell with the second light emitted from the light emitting layer; and analyzing the cell from the acquired image.

### CROSS-REFERENCE TO RELATED APPLICATION(S)

This application claims the benefit under 35 USC § 119 of Korean Patent Application No. 10-2023-0193731 filed on Dec. 27, 2023, in the Korean Intellectual Property Office, the entire disclosures of which are hereby incorporated by reference for all purposes.

### BACKGROUND

### 1. Field

The present disclosure relates to a cell analysis method and a cell analysis system, and more particularly, to a cell analysis method and a cell analysis system, capable of analyzing biophysical and biochemical properties of a cell by imaging the cell by a nonchemical scheme.

### 2. Description of the Related Art

Conventionally, various technologies for analyzing cells have been disclosed. For example, Korean Unexamined Patent Publication No. 10-2011-0058715 has disclosed a flow cytometry method including adjusting cell populations, which are targeted by antibodies conjugated with fluorescent dyes having the same color, to exhibit different fluorescence intensities according to types of the antibodies, wherein the adjusting of the cell populations to exhibit the different fluorescence intensities is performed by adjusting amounts of the antibodies conjugated with the fluorescent dyes to be different according to the types of the antibodies. For another example, Korean Unexamined Patent Publication No. 10-2019-0006164 has disclosed a system for performing flow cytometry, the system including: a laser configured to produce laser radiation to illuminate a sample; at least one detector arranged to detect at least a portion of the radiation emitted from the sample in response to the illumination, and generate a time signal; and an analysis module connected to the detector, and configured to receive the time signal and perform statistical analysis of the signal based on a forward model to reconstruct an image of the sample, wherein the statistical analysis is used to refine a parameter of the model by minimizing a difference between the time signal generated by the at least one detector and a corresponding time signal predicted by the forward model.

Meanwhile, the flow cytometry may essentially require chemical labeling and cell lysis. However, the chemical labeling and the cell lysis may be inefficient, and may be difficult to be applied as a base technology. Especially in a case of the chemical labeling, properties of cells may be modified, and accuracy of data may be reduced due to immunofluorescence contaminants.

Accordingly, recently, quantitative phase image analysis has been commercialized as an alternative to the flow cytometry. However, the quantitative phase image analysis may have a limitation of analyzing only physical properties such as a size or a refractive index of a cell. For this reason, with the conventional quantitative phase image analysis, analysis of cell properties may be limited.

Accordingly, there is a need for a method capable of analyzing chemical properties of cells, such as a substance and an amount of the substance secreted by the cell, in addition to physical properties of the cells.

### SUMMARY

One technical object of the present disclosure is to provide a cell analysis method and a cell analysis system, which do not require chemical labeling and cell lysis in analyzing cell properties.

Another technical object of the present disclosure is to provide a cell analysis method and a cell analysis system, capable of analyzing biophysical properties of a cell.

Still another technical object of the present disclosure is to provide a cell analysis method and a cell analysis system, capable of analyzing biochemical properties of a cell.

Technical objects of the present disclosure are not limited to the technical objects described above.

To achieve the technical objects described above, the present disclosure provides a cell analysis method.

In a general aspect of the disclosure, a cell analysis method includes: providing a flow unit configured to provide a path in which a cell flows, the flow unit including a light emitting layer configured to, in response to being irradiated by a first light in a first wavelength band, emit a second light in a second wavelength band; irradiating the light emitting layer with the first light; emitting the second light from the light emitting layer in response to the irradiation of the first light; acquiring an image formed by reacting the cell with the second light emitted from the light emitting layer; and analyzing the cell from the acquired image.

The cell analysis method may further include learning the cell, wherein the learning may include: converting the acquired image into a grayscale image; segmenting the grayscale image into a plurality of segmentation images; imaging the cell in the segmentation image; learning the imaged cell by applying a model; and managing a result related to the learned cell in a database.

The segmenting may include segmenting at least a portion overlaps in the segmentation images obtained through the segmenting.

The managing may include: managing a biophysical result of the learned cell in a biophysical database; or managing a biochemical result of the learned cell in a biochemical database.

The analyzing may further include at least one of: extracting the biophysical database for the cell from the acquired image; extracting the biochemical database for the cell from the acquired image; or a combination thereof.

The biophysical database may include at least one of a size, a shape, a refractive index of the cell, or any combination thereof, and the biochemical database may include a nitric oxide efflux rate of the cell.

The number of cells flowing in the flow unit may be between 10⁵ and less than 10⁷ per minute.

The first light may include a visible light, and the second light may include a near-infrared light.

The flow unit may further include an adhesive layer including collagen. For this reason, when the cell flows inside the flow unit, the cell can adhere to the adhesive layer.

For this reason, the cell adhering to the adhesive layer can be easily irradiated with the second light, and accuracy of the analysis of the cell can be improved.

In another general aspect of the disclosure, a cell analysis system includes: a flow unit configured to provide a path in which a cell flows, the flow unit including a light emitting layer configured to, in response to being irradiated by a first light in a first wavelength band, emit a second light in a second wavelength band; an acquisition unit configured to acquire an image formed by reacting the cell with the second light; and an analysis unit configured to analyze the cell from the image acquired by the acquisition unit.

The cell analysis system may further include an irradiation unit configured to irradiate the light emitting layer with the first light, wherein the irradiation unit may include: a laser configured to perform the irradiation of the first light; a diffuser lens configured to diffuse the first light subjected to the irradiation by the laser; and a convex lens configured to collect the first light diffused by the diffuser lens.

The acquisition unit may include: a stage on which the flow unit is seated, the stage including a transmission layer configured to transmit the first light through the seated flow unit; a beam splitter configured to reflect the first light subjected to the irradiation by the irradiation unit toward the flow unit seated on the stage; an objective lens configured to collect the first light reflected by the beam splitter to provide the collected first light to the light emitting layer on the stage; and a camera configured to form an image from a reaction signal of the cell with the second light emitted from the light emitting layer in response to the irradiation of the first light.

The acquisition unit and the convex lens may have at least one of: a first separation distance of between 8 cm and 12 cm; a second separation distance of between 30.5 cm and 34.5 cm; or a third separation distance of between 39 cm and 43 cm.

The laser may be configured to perform the irradiation of the first light having a power of between 100 mW and 500 mW in a wavelength band of between 560 nm and 750 nm, wherein the convex lens may have a diameter of between 30 mm and 70 mm, and a focal length of between 80 mm and 120 mm, and wherein the diffuser lens may have a diameter of between 5 mm and 45 mm, and a grit between 100 mm and 140 mm.

For the first to third separation distances, a magnification power of the objective lens may be greater than a magnification power of between 5 and 20.

For the first to third separation distances, a number of cells flowing in the flow unit may be between 10⁵ and 10⁷ per minute.

The cell analysis system may further comprise a learning unit configured to learn the cell, wherein the learning unit may be configured to: learn the cell by segmenting the acquired image into a plurality of segmentation images, such that at least a portion overlaps in the segmentation images obtained through the segmenting; and manage a result related to the learned cell in a database.

The learning unit may be further configured to: manage a biophysical result of the learned cell in a biophysical database; or manage a biochemical result of the learned cell in a biochemical database.

The analysis unit may be further configured to: extract the biophysical database managed by the learning unit for the cell of the acquired image; or extract the biochemical database managed by the learning unit for the cell of the acquired image.

The biophysical database may include at least one of a size, a shape, a refractive index of the cell, or any combination thereof, and the biochemical database may include a nitric oxide efflux rate of the cell.

The first light may include a visible light, and the second light may include a near-infrared light.

For the reason(s) above, the number of cells included in the image acquired by the acquisition unit can be further maximized, and the accuracy of the analysis of the cell can be further maximized.

### BRIEF DESCRIPTION OF THE DRAWINGS

FIG. 1 is a view for describing a cell analysis system according to an embodiment of the present disclosure.

FIGS. 2 and 3 are views for describing a cell analysis method according to an embodiment of the present disclosure.

FIG. 4 is a view for describing a flow unit according to the embodiment of the present disclosure.

FIG. 5 is a view for describing an irradiation unit according to the embodiment of the present disclosure.

FIG. 6 is a view for describing an acquisition unit according to the embodiment of the present disclosure.

FIG. 7 is a view for describing an analysis unit and a learning unit according to the embodiment of the present disclosure.

FIG. 8 is a view for describing a method for acquiring data of a cell according to the embodiment of the present disclosure.

FIG. 9 is a view for describing a learning method of the learning unit according to the embodiment of the present disclosure.

FIG. 10 is a near-infrared image of a cell acquired with settings of the irradiation unit and the acquisition unit according to Experimental Example 1-1 of the present disclosure.

FIG. 11 is a near-infrared image of a cell acquired with settings of the irradiation unit and the acquisition unit according to Experimental Examples 1-2 to 1-7 of the present disclosure.

FIG. 12 is a near-infrared image of a cell acquired with settings of the irradiation unit and the acquisition unit according to Experimental Example 2-1 of the present disclosure.

FIG. 13 is a near-infrared image of a cell acquired with settings of the irradiation unit and the acquisition unit according to Experimental Example 2-2 of the present disclosure.

FIG. 14 is a near-infrared image of a cell acquired with settings of the irradiation unit and the acquisition unit according to Experimental Example 2-3 of the present disclosure.

FIG. 15 is a near-infrared image of a cell acquired under a cell injection condition according to Experimental Example 3-1 of the present disclosure and a bounding box marked on the near-infrared image.

FIG. 16 is a near-infrared image of a cell acquired under a cell injection condition according to Experimental Example 3-2 of the present disclosure and a bounding box marked on the near-infrared image.

FIG. 17 is a near-infrared image of a cell acquired under a cell injection condition according to Experimental Example 3-3 of the present disclosure and a bounding box marked on the near-infrared image.

FIG. 18 is a view for describing a learning method of a learning unit according to an experimental example of the present disclosure.

FIGS. 19 and 20 are views for describing an analysis method of an analysis unit according to the experimental example of the present disclosure.

FIG. 21 is a view for describing immune heterogeneity of human monocyte reactive oxygen species (ROS), which is analyzed through a cell analysis system according to the experimental example of the present disclosure.

FIG. 22 is a view for describing heterogeneity of inducible nitric oxide synthase (iNOS) of a giant cell of a mouse, which is analyzed through the cell analysis system according to the experimental example of the present disclosure.

FIG. 23 is a view for describing the learning unit according to the experimental example of the present disclosure.

FIG. 24 is a view for describing a model according to the experimental example of the present disclosure.

FIG. 25 is a view for describing a variation curve of an F1 score according to a confidence threshold according to the experimental example of the present disclosure.

FIG. 26 is a graph showing learning curves and losses thereof of the model according to the experimental example of the present disclosure.

FIG. 27 is a graph showing relation between precision and recall of the model according to the experimental example of the present disclosure.

FIG. 28 is a view for describing an imaging result of a cell according to the experimental example of the present disclosure.

FIG. 29 shows an imaging result to which a result of performing learning by the learning unit is applied according to the experimental example of the present disclosure.

FIG. 30 is a view for describing relation among a size, a refractive index, and a full width at half maximum (FWHM) of the cell according to the experimental example of the present disclosure.

FIG. 31 is a view for describing sizes, refractive indexes, and photonic nanojets of various cells according to the experimental example of the present disclosure.

FIGS. 32A and 32B illustrate a near-infrared image and a full width at half maximum (FWHM) calculation result of a passage number 5 (P5) cell in a human dermal fibroblast (HDF) according to the experimental example of the present disclosure.

FIGS. 33A and 33B illustrate a near-infrared image and a full width at half maximum (FWHM) calculation result of a passage number 15 (P15) cell in a human dermal fibroblast (HDF) according to the experimental example of the present disclosure.

FIG. 34 is a graph showing refractive indexes of the P5 and P15 cells in the human dermal fibroblast (HDF) according to the experimental example of the present disclosure.

FIG. 35 is a view showing a state in which an activated macrophage (Activated) and a nonactivated macrophage (Nonactivated) are spatially separated from a background in a near-infrared image according to the experimental example of the present disclosure.

FIG. 36 is a graph showing relation between an intensity and an area of the activated macrophage (Activated) and the nonactivated macrophage (Nonactivated) according to the experimental example of the present disclosure.

FIG. 37 is a graph showing area distribution of the activated macrophage (Activated) and the nonactivated macrophage (Nonactivated) according to the experimental example of the present disclosure.

FIG. 38 is a graph showing intensity distribution of the activated macrophage (Activated) and the nonactivated macrophage (Nonactivated) according to the experimental example of the present disclosure.

### DETAILED DESCRIPTION

Hereinafter, exemplary embodiments of the present disclosure will be described in detail with reference to the accompanying drawings. However, the technical idea of the present disclosure is not limited to the embodiments described herein, but may be embodied in different forms. The embodiments introduced herein are provided to sufficiently deliver the idea of the present disclosure to those skilled in the art so that the disclosed contents may become thorough and complete.

When it is mentioned in the present disclosure that one element is on another element, it means that one element may be directly formed on another element, or a third element may be interposed between one element and another element. Further, in the drawings, thicknesses of films and regions are exaggerated for effective description of the technical contents.

In addition, although the terms such as first, second, and third have been used to describe various elements in various embodiments of the present disclosure, the elements are not limited by the terms. The terms are used only to distinguish one element from another element. Therefore, an element mentioned as a first element in one embodiment may be mentioned as a second element in another embodiment. The embodiments described and illustrated herein include their complementary embodiments, respectively. Further, the term “and/or” used in the present disclosure is used to include at least one of the elements enumerated before and after the term.

As used herein, an expression in a singular form includes a meaning of a plural form unless the context clearly indicates otherwise. Further, the terms such as “including” and “having” are intended to designate the presence of features, numbers, steps, elements, or combinations thereof described herein, and shall not be construed to preclude any possibility of the presence or addition of one or more other features, numbers, steps, elements, or combinations thereof. In addition, the term “connection” used herein is used to include both indirect and direct connections of a plurality of elements.

Further, in the following description of the present disclosure, detailed descriptions of known functions or configurations incorporated herein will be omitted when they may make the gist of the present disclosure unnecessarily unclear.

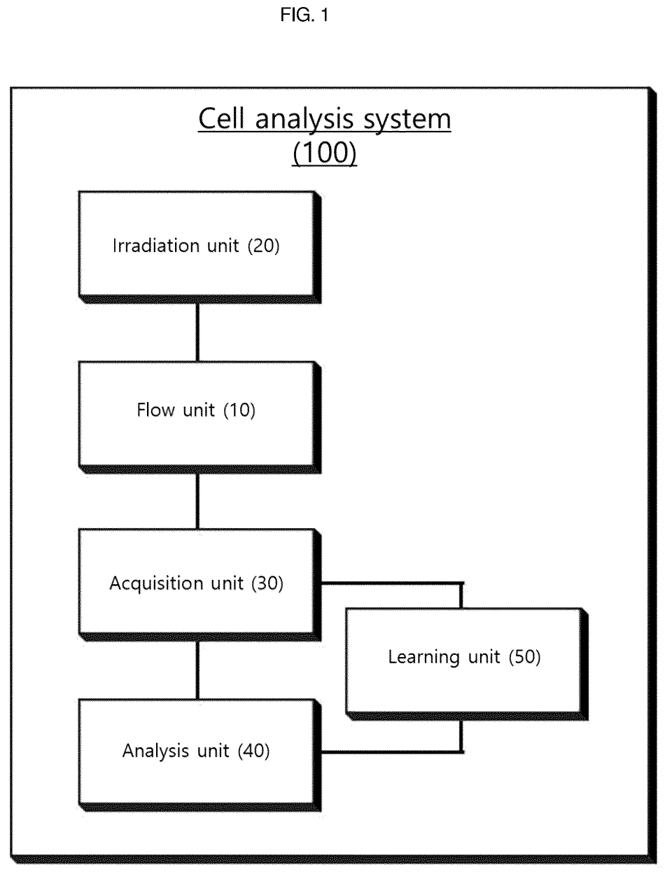

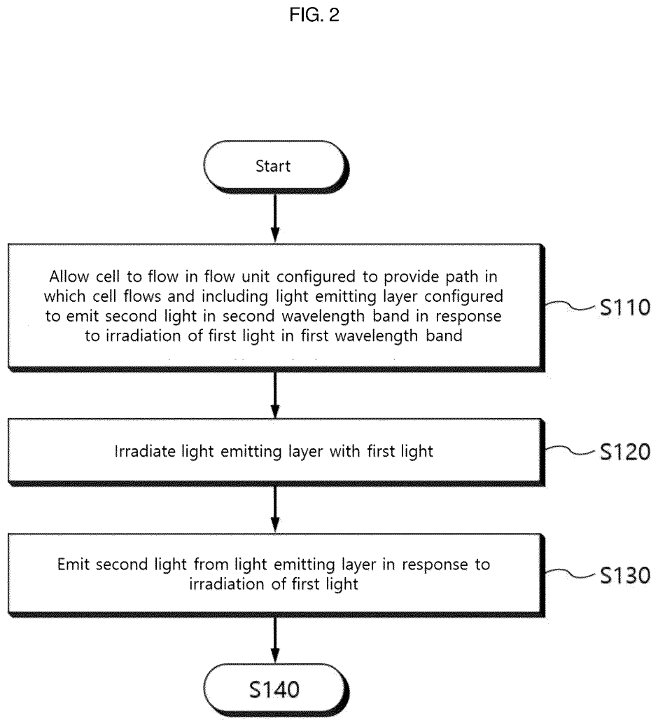

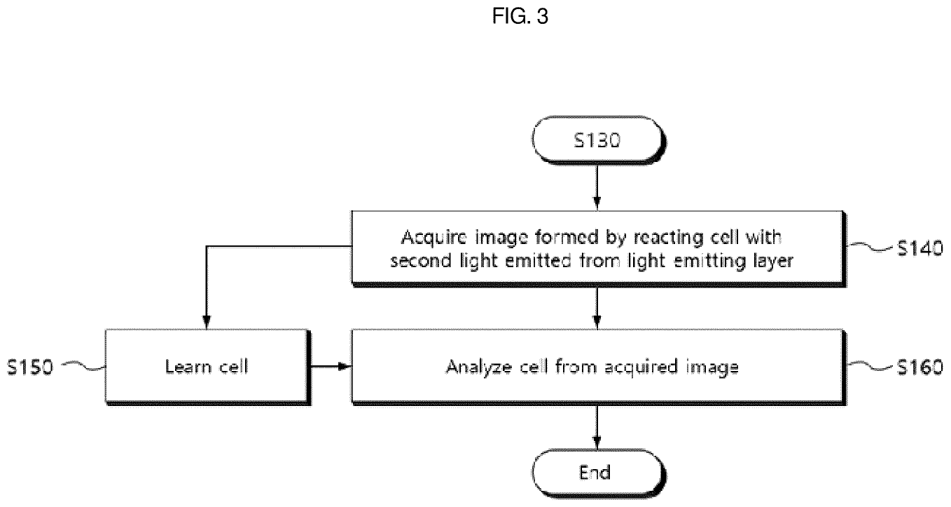

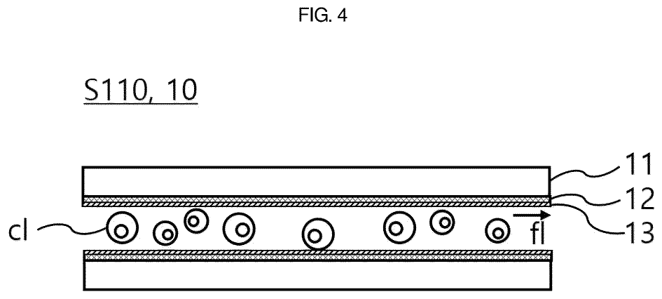

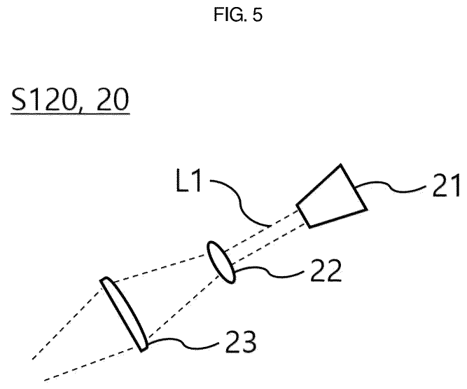

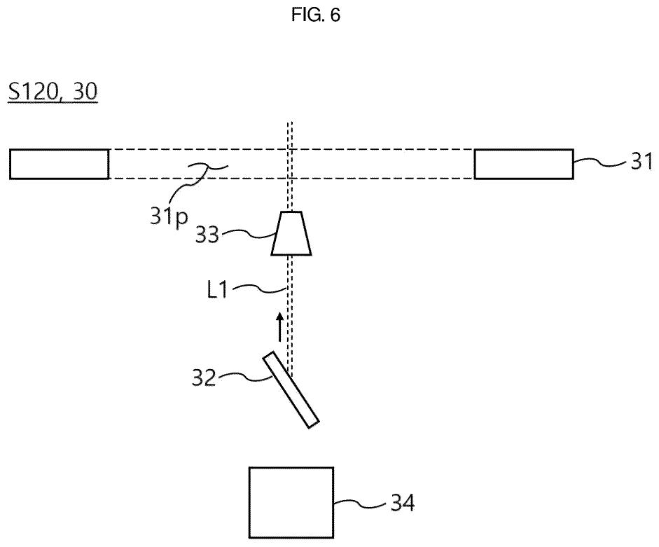

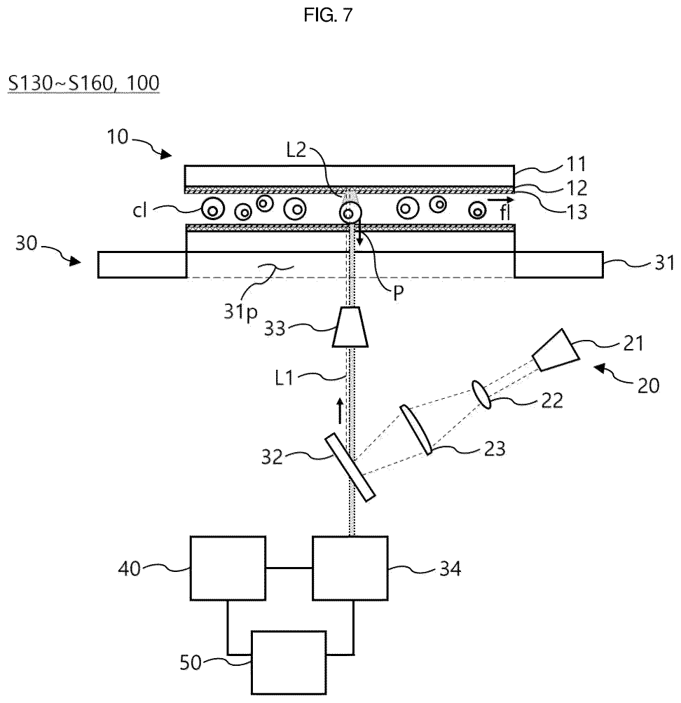

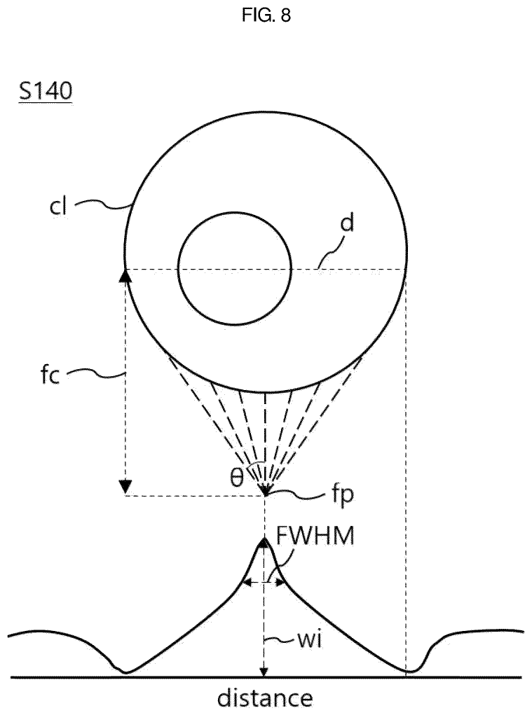

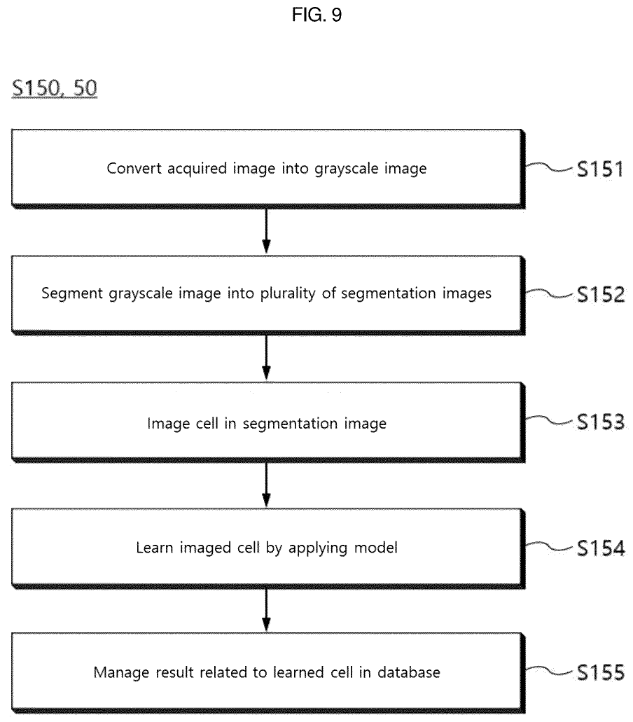

FIG. 1 is a view for describing a cell analysis system according to an embodiment of the present disclosure, FIGS. 2 and 3 are views for describing a cell analysis method according to an embodiment of the present disclosure, FIG. 4 is a view for describing a flow unit according to the embodiment of the present disclosure, FIG. 5 is a view for describing an irradiation unit according to the embodiment of the present disclosure, FIG. 6 is a view for describing an acquisition unit according to the embodiment of the present disclosure, FIG. 7 is a view for describing an analysis unit and a learning unit according to the embodiment of the present disclosure, FIG. 8 is a view for describing a method for acquiring data of a cell according to the embodiment of the present disclosure, and FIG. 9 is a view for describing a learning method of the learning unit according to the embodiment of the present disclosure.

Referring to FIG. 1, a cell analysis system 100 may include at least one of: a flow unit 10 configured to provide a path in which a cell cl (see FIG. 4) flows (fl, see FIG. 4), and including a light emitting layer 12 (see FIG. 7) configured to emit a second light L2 (see FIG. 7) in a second wavelength band in response to irradiation of a first light L1 (see FIG. 5) in a first wavelength band; an irradiation unit 20 configured to irradiate the light emitting layer 12 with the first light L1; an acquisition unit 30 configured to acquire an image formed by reacting the cell cl with the second light L2; and an analysis unit 40 configured to analyze the cell cl from the image acquired by the acquisition unit 30; and a learning unit 50 configured to learn the cell cl.

In more detail, referring to FIGS. 2 and 4, a cell cl may flow in a flow unit 10 configured to provide a path in which the cell cl flows (fl) and including a light emitting layer 12 configured to emit a second light L2 in a second wavelength band in response to irradiation of a first light L1 in a first wavelength band (S110).

According to one embodiment, as shown in FIG. 4, the flow unit 10 may include a channel 11, the light emitting layer 12 formed on an inner wall of the channel 11, and an adhesive layer 13 formed on the light emitting layer 12. In more detail, the light emitting layer 12 may include a single-wall carbon nanotube (SWNT). For this reason, the light emitting layer 12 may emit the second light L2 in the second wavelength band in response to the irradiation of the first light L1 in the first wavelength band. For example, the first light L1 may be a visible light, and the second light L2 may be a near-infrared light, more specifically, a near-infrared fluorescent light. In other words, the light emitting layer 12 may emit the second light L2, that is, the near-infrared fluorescent light, upon the irradiation of the first light L1, that is, the visible light.

According to one embodiment, the flow unit 10 may include: an inlet (not shown) through which the cell cl is introduced; and an outlet (not shown) through which the introduced cell cl is discharged. In more detail, the channel 11 may have a shape of a conduit in which the cell cl may flow (fl), and the inlet and the outlet may be formed at both ends of the channel 11, respectively. For this reason, when a liquid including the cell cl is introduced through the inlet, the liquid including the cell cl may pass through a conduit inside the channel 11 so as to be discharged through the outlet. In other words, according to an embodiment of the present disclosure, the flow unit 10 may provide fluidity (fl) to the cell cl.

According to one embodiment, the adhesive layer 13 may include collagen. For this reason, the adhesive layer 13 may have excellent adhesion to the cell cl flowing (fl) inside the channel 11. In other words, when the cell cl flows inside the flow unit 10, that is, the channel 11, the cell cl may adhere to the adhesive layer 13.

For this reason, the cell cl adhering to the adhesive layer 13 may be easily irradiated with the second light L2, and accuracy of the analysis of the cell cl may be improved.

Next, referring to FIGS. 2, 5, and 6, the light emitting layer 12 may be irradiated with the first light L1 (S120).

According to one embodiment, as shown in FIG. 5, the irradiation unit 20 may include: a laser 21 configured to perform the irradiation of the first light L1; a diffuser lens 22 configured to diffuse the first light L1 subjected to the irradiation by the laser 21; and a convex lens 23 configured to collect the first light L1 diffused by the diffuser lens 22. In detail, for example, referring to FIG. 7, the convex lens 23 may have a first separation distance of more than 8 cm and less than 12 cm from a beam splitter 32 of the acquisition unit 30, the diffuser lens 22 may have a second separation distance of more than 30.5 cm and less than 34.5 cm from the beam splitter 32 of the acquisition unit 30, and the laser 21 may have a third separation distance of more than 39 cm and less than 43 cm from the beam splitter 32 of the acquisition unit 30. In more detail, for example, the convex lens 23 may have a first separation distance of 10 cm from the beam splitter 32 of the acquisition unit 30, the diffuser lens 22 may have a second separation distance of 32.5 cm from the beam splitter 32 of the acquisition unit 30, and the laser 21 may have a third separation distance of 41 cm from the beam splitter 32 of the acquisition unit 30.

For this reason, the image of the cell cl acquired by the acquisition unit 30 may be clear, so that accuracy of the analysis of the cell cl may be improved.

In addition, in detail, for example, the laser 21 may perform the irradiation of the first light L1 having a power of 100 mW or more and 500 mW or less in a wavelength band of 560 nm or more and 750 nm or less, the convex lens 23 may have a diameter of 30 mm or more and 70 mm or less and a focal length of 80 mm or more and 120 mm or less, and the diffuser lens 22 may have a diameter of 5 mm or more and 45 mm or less and a grit of 100 or more and 140 mm or less. In more detail, for example, the laser 21 may perform the irradiation of the first light L1 having a power of 300 mW in a wavelength band of 721 nm, the convex lens 23 may have a diameter of 50 mm and a focal length of 100 mm, and the diffuser lens 22 may have a diameter of 25 mm and a grit of 120. In this case, in detail, for example, while the irradiation unit 20 has the first to third separation distances, the number of cells cl flowing in the flow unit 10 may be greater than 10⁵ and less than 10⁷ per minute. In more detail, for example, while the irradiation unit 20 has the first to third separation distances, the number of cells cl flowing in the flow unit 10 may be 106 per minute.

For this reason, the number of cells cl included in the image acquired by the acquisition unit 30 may be maximized, and the accuracy of the analysis of the cell cl may be maximized.

Alternatively, for another example, the laser 21 may perform the irradiation of the first light L1 having a power of 100 mW or more and 500 mW or less in a wavelength band of 561 nm. In other words, according to the embodiment of the present disclosure, the wavelength band of the first light L1 subjected to the irradiation by the laser 21 is not limited.

Alternatively, for another example, the convex lens 23 may have a diameter of 30 mm or more and 70 mm or less and a focal length of 75 mm, 100 mm, and/or 150 mm. In other words, according to the embodiment of the present disclosure, the focal length of the convex lens 23 is not limited.

Alternatively, for another example, the diffuser lens 22 may have a diameter of 5 mm or more and 45 mm or less and a grit of 200 and/or 600. In other words, according to the embodiment of the present disclosure, the grit of the diffuser lens 22 is not limited.

Alternatively, for another example, while the irradiation unit 20 has the first to third separation distances, the number of cells cl flowing in the flow unit 10 may be 10⁵ or less and/or 10⁷ per minute. In other words, according to the embodiment of the present disclosure, the number of cells cl flowing in the flow unit 10 is not limited.

According to one embodiment, as shown in FIG. 6, the acquisition unit 30 may include: a stage 31 on which the flow unit 10 is seated, and including a transmission layer 31p configured to transmit the first light L1 to the seated flow unit 10; a beam splitter 32 configured to reflect the first light L1 subjected to the irradiation by the irradiation unit 20 toward the flow unit 10 seated on the stage 31; an objective lens 33 configured to collect the first light L1 reflected by the beam splitter 32 to provide the collected first light L1 to the light emitting layer 12 on the stage 31; and a camera 34 configured to form an image from a reaction signal of the cell cl with the second light L2 emitted from the light emitting layer 12 in response to the irradiation of the first light L1. In detail, for example, while the irradiation unit 20 has the first to third separation distances, a magnification power of the objective lens 33 may be greater than a magnification power of 5 and less than a magnification power of 20. In more detail, for example, while the irradiation unit 20 has the first to third separation distances, a magnification power of the objective lens 33 may be a magnification power of 10.

For this reason, the number of cells cl included in the image acquired by the acquisition unit 30 may be further maximized, and the accuracy of the analysis of the cell cl may be further maximized.

Next, referring to FIGS. 2 and 7, the second light L2 may be emitted from the light emitting layer 12 in response to the irradiation of the first light L1 (S130).

According to one embodiment, the light emitting layer 12 may include a single-wall carbon nanotube (SWNT). For this reason, the light emitting layer 12 may emit the second light L2 upon the irradiation of the first light L1. For example, the first light L1 may be a visible light, and the second light L2 may be a near-infrared light, more specifically, a near-infrared fluorescent light.

Next, referring to FIGS. 3 and 7, an image formed by reacting the cell cl with the second light L2 emitted from the light emitting layer 12 may be acquired (S140). In more detail, the cell cl may interact with the second light L2, that is, the near-infrared fluorescent light, emitted from the light emitting layer 12. In more detail, when the cell cl interacts with the second light L2, that is, the near-infrared fluorescent light, emitted from the light emitting layer 12, a photonic nanojet P may be generated by the interaction. When the photonic nanojet P is generated, the cell cl may focus the second light L2 through the photonic nanojet P like an optical lens. For this reason, an image for the cell cl may be acquired by using the second light L2 focused by the cell cl as a signal. In more detail, the image may be a near-infrared image.

The near-infrared image of the cell cl may include biophysical data and biochemical data of the cell cl. In other words, when the near-infrared image of the cell cl is acquired, the biophysical data and the biochemical data of the cell cl may be acquired. In more detail, referring to FIG. 8, when the photonic nanojet P is generated, and the cell cl focuses the second light L2 through the photonic nanojet P, at least one of a focal point fp, a focal length fc, a maximum wavelength intensity wi, and a full width at half maximum (FWHM) of the focused second light L2 may be acquired as a numerical value.

For this reason, the biophysical data of the cell cl as well as the biochemical data of the cell cl may be acquired. The biophysical data may include at least one of a size, a shape, and a refractive index of the cell cl. The biochemical data may include a nitric oxide efflux rate of the cell cl.

In other words, according to the embodiment of the present disclosure, when the near-infrared image of the cell cl is acquired, biophysical and biochemical heterogeneity of the cell cl, such as information on a protein, a ribonucleic acid (RNA) transcriptional expression degree, and/or a migration of the cell cl, may be evaluated.

Furthermore, when the near-infrared image of the cell cl is acquired, the biophysical data and the biochemical data of the cell cl may be managed in a database.

Next, referring to FIGS. 3 and 7, the cell cl may be learned (S150). In more detail, referring to FIG. 9, in order to learn the cell cl, first, the acquired image may be converted into a grayscale image (S151). In more detail, the acquired image may be converted into the grayscale image after an average pixel value is calculated.

According to one embodiment, the grayscale image may be adjusted through adaptive histogram equalization. In addition, a background brightness of the grayscale image may be adjusted to be uniform.

For this reason, data acquired from the grayscale image may be uniform.

Next, referring to FIG. 9, the grayscale image may be segmented into a plurality of segmentation images (S152). In more detail, the grayscale image may be subjected to the segmenting such that at least a portion overlaps in the segmentation images obtained through the segmenting. For example, when the grayscale image is segmented into patches, each patch having 100×100 pixels, an overlapping portion having 20 pixels may exist between the patches.

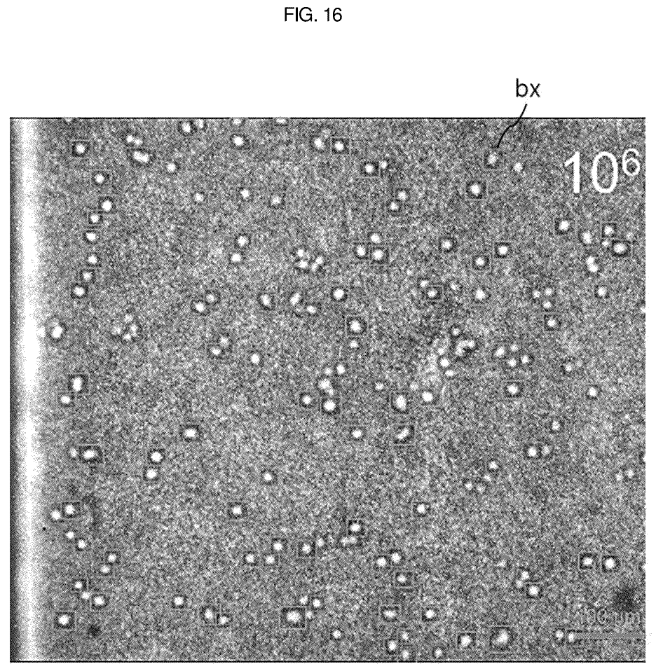

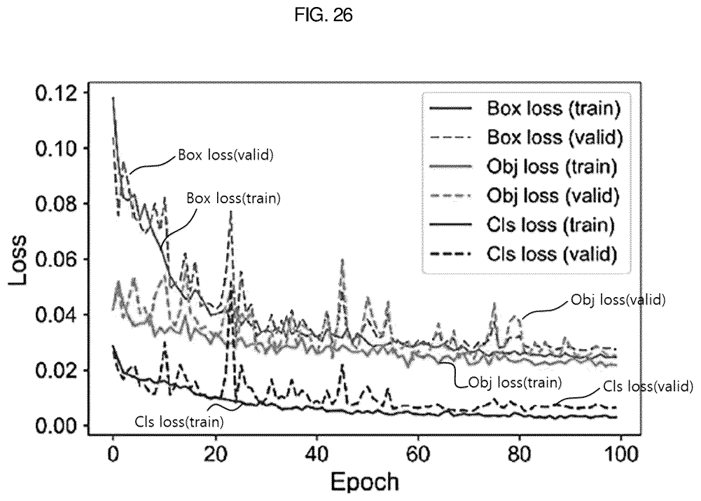

Next, referring to FIG. 9, the cell cl may be imaged in the segmentation image (S153). According to the present disclosure, the imaging may be different from conventional chemical labeling that may modify cell properties and reduce accuracy of data due to immunofluorescence contaminants. In more detail, the imaging may include annotating each of the cells cl included in the segmentation image, and distinguishing each of the cells cl with a bounding box bx (see FIGS. 16 and 26). The imaging may be, for example, YOLO imaging performed by using an imaging tool. However, embodiments are not limited thereto. In other words, the imaging may refer to a method for imaging the cell cl by a nonchemical scheme.

For this reason, according to the embodiment of the present disclosure, unlike the conventional chemical labeling, modification of properties of the cell cl may be minimized, and immunofluorescence contaminants may be minimized, so that accuracy of data may be excellent.

Next, referring to FIG. 9, the imaged cell cl may be learned by applying a model (S154). In more detail, the model may be applied to the imaged cell cl for deep learning. The model may be, for example, YOLOv5. However, embodiments are not limited thereto. The YOLOv5 may include a backbone, a neck, and a head. Cross stage hierarchical networks may be used in the backbone, and a path aggregation network (PANet) may be used in the neck. However, embodiments are not limited thereto. The head may detect an object, localize a bounding box, evaluate object confidence, and perform classification.

According to one embodiment, the learning of the cell cl to which the model is applied may be repeatedly performed. For example, the learning of the cell cl to which the model is applied may be stopped when reduction of a loss does not occur for 50 consecutive epochs after the learning is performed for 300 epochs. However, embodiments are not limited thereto.

According to one embodiment, after the learning of the cell cl to which the model is applied, additional learning may be performed through an adaptive moment estimation (ADAM) optimization technique.

Next, referring to FIG. 9, a result related to the learned cell cl may be managed in a database (S155). In more detail, a biophysical result of the learned cell cl may be managed in a biophysical database. The biophysical database may include at least one of a size, a shape, and a refractive index of the cell cl. Alternatively, a biochemical result of the learned cell cl may be managed in a biochemical database. The biochemical database may include a nitric oxide efflux rate of the cell cl.

Meanwhile, the learning of the cell cl and the management of the database as described above may be performed by the learning unit 50. In detail, the learning unit 50 may learn the cell cl by segmenting the acquired image into a plurality of segmentation images, such that at least a portion overlaps in the segmentation images obtained through the segmenting, and manage a result related to the learned cell cl in a database. In more detail, the learning unit 50 may manage a biophysical result of the learned cell cl in a biophysical database, or manage a biochemical result of the learned cell cl in a biochemical database.

Next, referring to FIGS. 3 and 7, the cell cl may be analyzed from the acquired image (S160). In more detail, the analysis unit 40 may extract the biophysical database managed by the learning unit 50 for the cell cl of the acquired image, or extract the biochemical database managed by the learning unit 50 for the cell cl of the acquired image. The biophysical database may include at least one of a size, a shape, and a refractive index of the cell cl, and the biochemical database may include a nitric oxide efflux rate of the cell cl.

In other words, according to the embodiment of the present disclosure, when the near-infrared image of the cell cl is acquired, biophysical and biochemical heterogeneity of the cell cl, such as information on a protein, a ribonucleic acid (RNA) transcriptional expression degree, and/or a migration of the cell cl, may be evaluated.

Referring again to FIG. 3, in a case where the learning unit 50 continuously performs the learning of the cell cl (S150) to accumulate and manage the biophysical database and the biochemical database of the learned cell cl, when the image of the cell cl is acquired by the acquisition unit 30 (S140), accuracy of extracting the biophysical database and the biochemical database of the cell cl, which are managed by the learning unit 50, for the cell cl of the acquired image by the analysis unit 40 may be improved. In other words, the accuracy of extracting the database related to the cell cl of the acquired image may be improved. Furthermore, a speed at which the database related to the cell cl of the acquired image is extracted may be increased. In other words, according to the embodiment of the present disclosure, the cell cl may be analyzed rapidly and accurately.

As described above, according to the embodiment of the present disclosure, the cell cl may be subjected to the imaging so as to be analyzed.

For this reason, according to the embodiment of the present disclosure, unlike the conventional chemical labeling, the cell cl may be analyzed nonchemically, so that the modification of the properties of the cell cl may be minimized, and the immunofluorescence contaminants may be minimized, and thus the accuracy of the data may be excellent.

Hereinafter, specific experimental examples and property evaluation results according to the embodiment of the present disclosure will be described.

### Preparation of Flow Unit (10) According to Experimental Example

A single-stranded DNA (ssDNA) with a 30-base (dAdT) sequence and a single-wall carbon nanotube (SWNT) were prepared, and were suspended in a 0.1 M sodium chloride (NaCl) solution such that a mass ratio of ssDNA (2 mg/mL)/SWNT becomes 2:1, so that a SWNT/DNA solution was prepared. The SWNT/DNA solution was sonicated for 10 minutes at a power of 10 W by using a 3 mm probe tip, and sonicated for 1 minute at a 40% amplitude in an ice bath. Thereafter, the sonicated solution was centrifuged twice for 90 minutes at 16 rcf and 100 rcf, and a supernatant was collected as a nanosensor dispersion. A concentration of the collected nanosensor dispersion was estimated to be 10 to 80 mg/L through an extinction coefficient s 632 nm=0.036 (mg/L)−1.

In addition, ibiTreat (μ-Slide VI 0.1) was prepared as the channel 11, and the channel 11 and 2 μL of 3-aminopropyl triethoxysilane (APTES) were immersed in ethanol (1% APTES, 1% water (H₂O)) and cultured for 3 hours.

After an inner wall of the cultured channel 11 is provided and coated with silane, 2 μL of the nanosensor dispersion was injected. After the nanosensor dispersion is evaporated on the inner wall of the channel 11 coated with the silane overnight, a surface of the channel 11 was rinsed twice with 1 mL of phosphate-buffered saline (PBS, pH 7.4) to remove the nanosensor dispersion that was not bound to the channel 11, so that the light emitting layer 12 was formed on the inner wall of the channel 11.

A collagen solution was injected into the inner wall of the channel 11 on which the light emitting layer 12 is formed, and cultured in a humidified chamber at 37° C., so that the adhesive layer 13 having adhesion to the cells cl was formed on the light emitting layer 12.

The inner wall of the channel 11 on which the light emitting layer 12 is formed was washed with phosphate-buffered saline (PBS) to remove the nanosensor dispersion and the collagen, which were not bound, so that the flow unit 10 according to the experimental example was prepared.

### Injection of Cells (Cl) According to Experimental Example

Macrophages (Raw 264.7 cells (ATCC TIB-71)) were injected into the flow unit 10 prepared according to the experimental example described above by using a syringe pump at 0.1 to 10 μL/min, and cultured for 3 days. In order to activate the macrophages, 10 μL of lipopolysaccharides from Escherichia coli O55:B5 (LPS (Escherichia coli O55:B5)) was additionally injected into the flow unit 10, and efflux of nitric oxide was induced.

Experimental examples that will be described below are methods and results of injecting the cells cl according to the experimental example into the flow unit 10 prepared according to the experimental example under set conditions, and acquiring, learning, and analyzing an image of the cells cl under respective set conditions.

### Settings of Irradiation Unit (20) and Acquisition Unit (30) According to Experimental Example 1-1 (Ex1-1)

The irradiation unit 20 including the laser 21 configured to perform irradiation of a visible light having a power of 300 mW and a wavelength band of 721 nm, a diffuser lens 22 having a diameter of 25 mm and a grit of 120, and a convex lens 23 having a diameter of 50 mm and a focal length of 100 mm was set.

The first separation distance between the convex lens 23 and the beam splitter 32 of the acquisition unit 30 was set to 10 cm, the second separation distance between the diffuser lens 22 and the beam splitter 32 was set to 32.5 cm, and the third separation distance between the laser 21 and the beam splitter 32 was set to 41 cm.

### Settings of Irradiation Unit (20) and Acquisition Unit (30) According to Experimental Example 1-2 (Ex1-2)

In Experimental Example 1-1 described above, the first separation distance between the convex lens 23 and the beam splitter 32 of the acquisition unit 30 was set to 8 cm, the second separation distance between the diffuser lens 22 and the beam splitter 32 was set to 32.5 cm, and the third separation distance between the laser 21 and the beam splitter 32 was set to 41 cm.

### Settings of Irradiation Unit (20) and Acquisition Unit (30) According to Experimental Example 1-3 (Ex1-3)

In Experimental Example 1-1 described above, the first separation distance between the convex lens 23 and the beam splitter 32 of the acquisition unit 30 was set to 12 cm, the second separation distance between the diffuser lens 22 and the beam splitter 32 was set to 32.5 cm, and the third separation distance between the laser 21 and the beam splitter 32 was set to 41 cm.

### Settings of Irradiation Unit (20) and Acquisition Unit (30) According to Experimental Example 1-4 (Ex1-4)

In Experimental Example 1-1 described above, the first separation distance between the convex lens 23 and the beam splitter 32 of the acquisition unit 30 was set to 10 cm, the second separation distance between the diffuser lens 22 and the beam splitter 32 was set to 30.5 cm, and the third separation distance between the laser 21 and the beam splitter 32 was set to 41 cm.

### Settings of Irradiation Unit (20) and Acquisition Unit (30) According to Experimental Example 1-5 (Ex1-5)

In Experimental Example 1-1 described above, the first separation distance between the convex lens 23 and the beam splitter 32 of the acquisition unit 30 was set to 10 cm, the second separation distance between the diffuser lens 22 and the beam splitter 32 was set to 34.5 cm, and the third separation distance between the laser 21 and the beam splitter 32 was set to 41 cm.

### Settings of Irradiation Unit (20) and Acquisition Unit (30) According to Experimental Example 1-6 (Ex1-6)

In Experimental Example 1-1 described above, the first separation distance between the convex lens 23 and the beam splitter 32 of the acquisition unit 30 was set to 10 cm, the second separation distance between the diffuser lens 22 and the beam splitter 32 was set to 32.5 cm, and the third separation distance between the laser 21 and the beam splitter 32 was set to 39 cm.

### Settings of Irradiation Unit (20) and Acquisition Unit (30) According to Experimental Example 1-7 (Ex1-7)

In Experimental Example 1-1 described above, the first separation distance between the convex lens 23 and the beam splitter 32 of the acquisition unit 30 was set to 10 cm, the second separation distance between the diffuser lens 22 and the beam splitter 32 was set to 32.5 cm, and the third separation distance between the laser 21 and the beam splitter 32 was set to 43 cm.

Experimental Examples 1-1 to 1-7 described above may be organized as shown in Table 1 below.

TABLE

| | First | | Second | Third |
|---|---|---|---|---|
| | Separation | | Separation | Separation |
| Classification | Distance | | Distance | Distance |
| Experimental | 10 | cm | 32.5 cm | 41 cm |
| Example 1-1 | | | | |
| (ex1-1) | | | | |
| Experimental | 8 | cm | 32.5 cm | 41 cm |
| Example 1-2 | | | | |
| (ex1-2) | | | | |
| Experimental | 12 | cm | 32.5 cm | 41 cm |
| Example 1-3 | | | | |
| (ex1-3) | | | | |
| Experimental | 10 | cm | 30.5 cm | 41 cm |
| Example 1-4 | | | | |
| (ex1-4) | | | | |
| Experimental | 10 | cm | 34.5 cm | 41 cm |
| Example 1-5 | | | | |
| (ex1-5) | | | | |
| Experimental | 10 | cm | 32.5 cm | 39 cm |
| Example 1-6 | | | | |
| (ex1-6) | | | | |
| Experimental | 10 | cm | 32.5 cm | 43 cm |
| Example 1-7 | | | | |

(ex1-7)

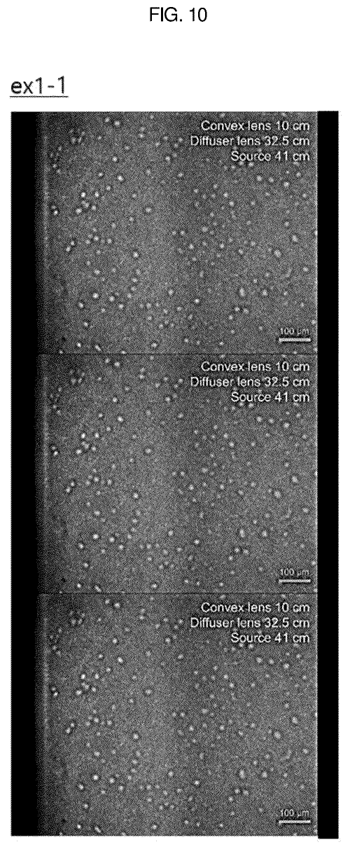

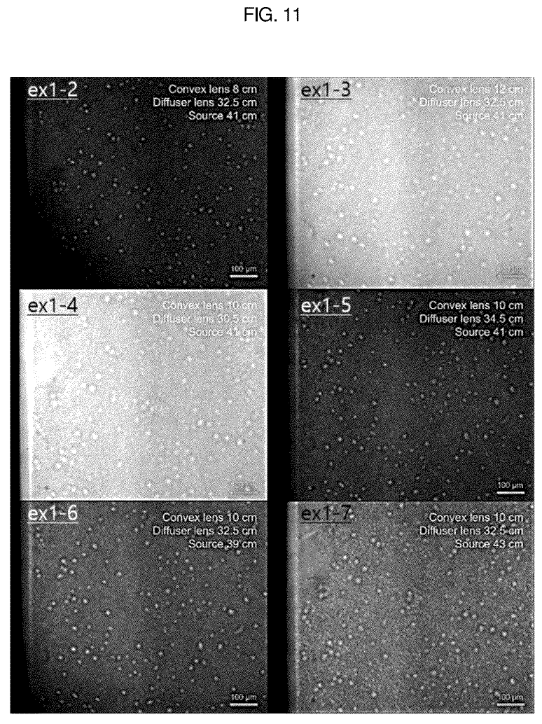

FIG. 10 is a near-infrared image of a cell acquired with settings of the irradiation unit and the acquisition unit according to Experimental Example 1-1 of the present disclosure, and FIG. 11 is a near-infrared image of a cell acquired with settings of the irradiation unit and the acquisition unit according to Experimental Examples 1-2 to 1-7 of the present disclosure.

Referring to FIGS. 10 and 11, the acquired near-infrared image of the cells cl was clearer in Experimental Example 1-1 than in Experimental Examples 1-2 to 1-7.

Accordingly, when the first separation distance between the convex lens 23 and the beam splitter 32 of the acquisition unit 30 is 10 cm, the second separation distance between the diffuser lens 22 and the beam splitter 32 is 32.5 cm, and the third separation distance between the laser 21 and the beam splitter 32 is 41 cm, the near-infrared image of the cells cl may be clear, so that it may be proven that the accuracy of the analysis of the cell cl may be improved.

### Settings of Irradiation Unit (20) and Acquisition Unit (30) According to Experimental Example 2-1 (Ex2-1)

In Experimental Example 1-1 described above, the magnification power of the objective lens 33 of the acquisition unit 30 was set to a magnification power of 5.

### Settings of Irradiation Unit (20) and Acquisition Unit (30) According to Experimental Example 2-2 (Ex2-2)

In Experimental Example 1-1 described above, the magnification power of the objective lens 33 of the acquisition unit 30 was set to a magnification power of 10.

### Settings of Irradiation Unit (20) and Acquisition Unit (30) According to Experimental Example 2-3 (Ex2-3)

In Experimental Example 1-1 described above, the magnification power of the objective lens 33 of the acquisition unit 30 was set to a magnification power of 20.

Experimental Examples 2-1 to 2-3 described above may be organized as shown in Table 2 below.

TABLE 2

| Classification | Magnification Power |
|---|---|
| Experimental Example 2-1 (ex2-1) | 5 |
| Experimental Example 2-2 (ex2-2) | 10 |
| Experimental Example 2-3 (ex2-3) | 20 |

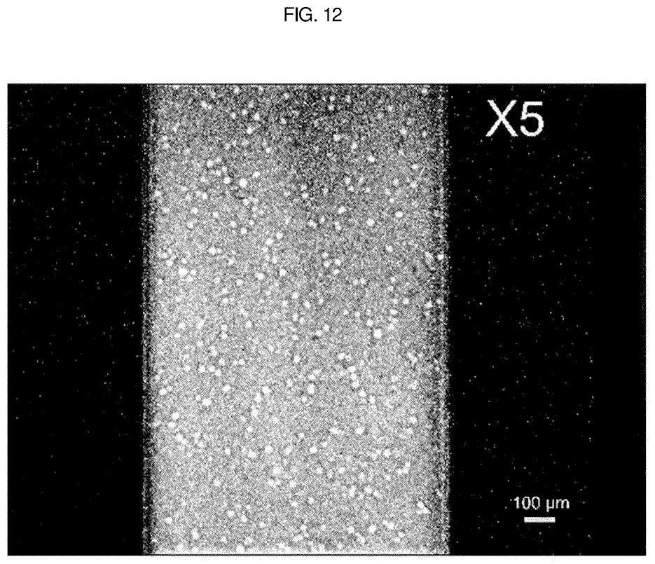

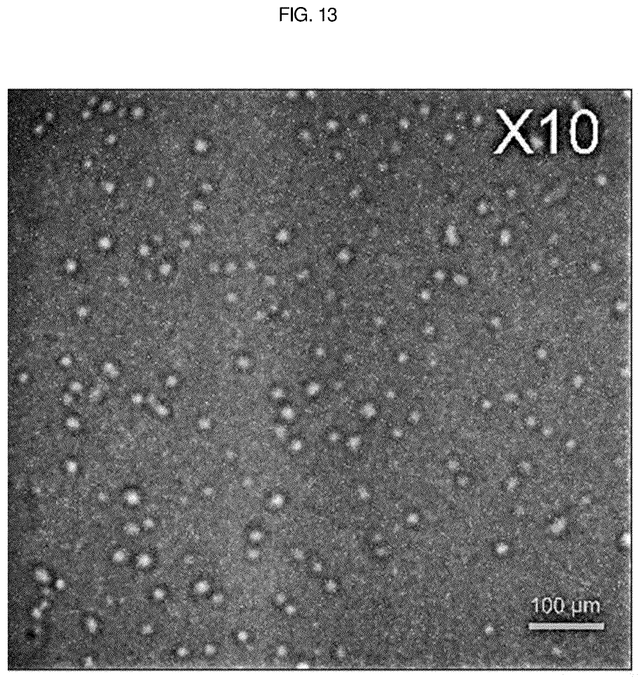

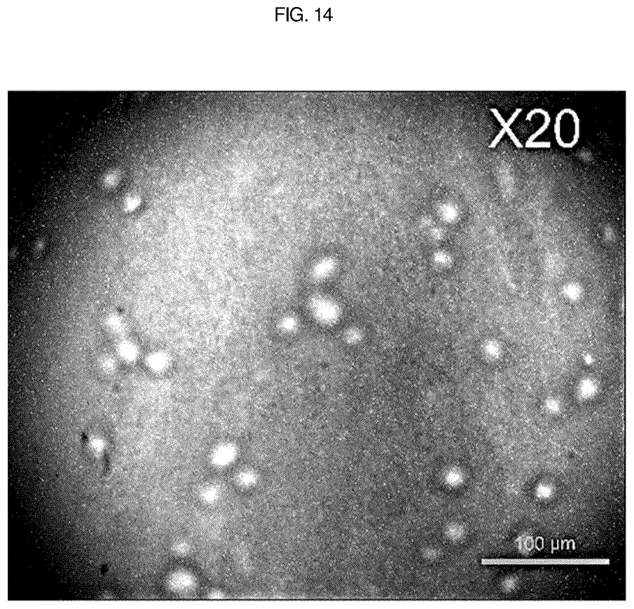

FIG. 12 is a near-infrared image of a cell acquired with settings of the irradiation unit and the acquisition unit according to Experimental Example 2-1 of the present disclosure, FIG. 13 is a near-infrared image of a cell acquired with settings of the irradiation unit and the acquisition unit according to Experimental Example 2-2 of the present disclosure, and FIG. 14 is a near-infrared image of a cell acquired with settings of the irradiation unit and the acquisition unit according to Experimental Example 2-3 of the present disclosure.

Referring to FIGS. 12 to 14, 110 to 170 cells cl were observed in the near-infrared image according to Experimental Example 2-1, 100 to 150 cells cl were observed in the near-infrared image according to Experimental Example 2-2, and 20 to 60 cells cl were observed in the near-infrared image according to Experimental Example 2-3, so that the largest number of cells cl were observed in Experimental Example 2-1.

However, when comparing Experimental Example 2-1 with Experimental Example 2-2, the cells cl were observed more clearly in Experimental Example 2-2 than in Experimental Example 2-1.

Accordingly, when the magnification power of the objective lens 33 is the magnification power of 10, it may be proven that the number of cells cl included in the near-infrared image may be maximized, and the accuracy of the analysis of the cell cl may be maximized.

### Injection of Cells According to Experimental Example 3-1 (Ex3-1)

In Experimental Example 2-2 described above, the cells cl were injected at 10⁵ cells per minute into the flow unit 10 to flow in the flow unit 10.

### Injection of Cells According to Experimental Example 3-2 (Ex3-2)

In Experimental Example 2-2 described above, the cells cl were injected at 106 cells per minute into the flow unit 10 to flow in the flow unit 10.

### Injection of Cells According to Experimental Example 3-3 (Ex3-3)

In Experimental Example 2-2 described above, the cells cl were injected at 10⁷ cells per minute into the flow unit 10 to flow in the flow unit 10.

Experimental Examples 2-1 to 2-3 described above may be organized as shown in Table 3 below.

TABLE 3

| Classification | Cell Injection Rate |
|---|---|
| Experimental Example 3-1 (ex3-1) | 10⁵ cells per minute |
| Experimental Example 3-2 (ex3-2) | 10⁶ cells per minute |
| Experimental Example 3-3 (ex3-3) | 10⁷ cells per minute |

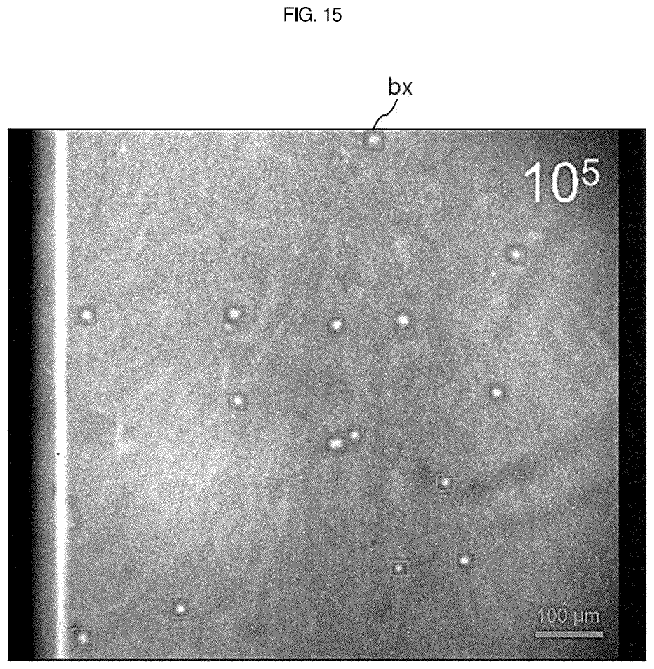

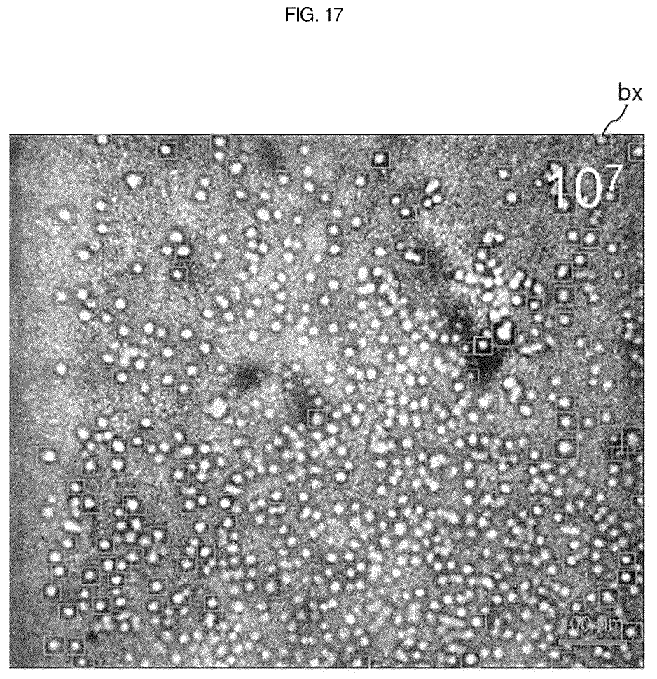

FIG. 15 is a near-infrared image of a cell acquired under a cell injection condition according to Experimental Example 3-1 of the present disclosure and a bounding box marked on the near-infrared image, FIG. 16 is a near-infrared image of a cell acquired under a cell injection condition according to Experimental Example 3-2 of the present disclosure and a bounding box marked on the near-infrared image, and FIG. 17 is a near-infrared image of a cell acquired under a cell injection condition according to Experimental Example 3-3 of the present disclosure and a bounding box marked on the near-infrared image.

Referring to FIGS. 15 to 17, in the near-infrared image according to Experimental Example 3-1, the imaging was performed well with the bounding box bx for each of the cells cl. However, in the near-infrared image according to Experimental Example 3-1, the number of cells cl included in one image was significantly small. Meanwhile, in the near-infrared image according to Experimental Example 3-2, the number of cells cl included in one image was appropriate, and the imaging was performed well with the bounding box bx for each of the cells cl. Meanwhile, in the near-infrared image according to Experimental Example 3-3, the number of cells cl included in one image was large, while the imaging was not performed well, which may be observed through presence of the cells cl that are not distinguished with the bounding box bx.

Accordingly, when the cells cl are injected at 106 cells per minute into the flow unit 10 to flow in the flow unit 10, it may be proven that the number of cells cl included in the near-infrared image may be maximized, and the accuracy of the analysis of the cell cl may be maximized.

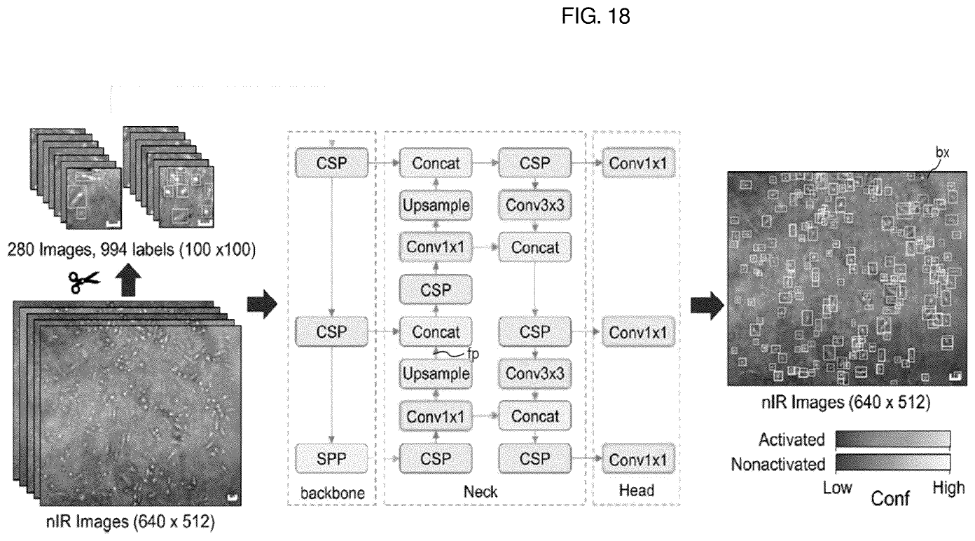

FIG. 18 is a view for describing a learning method of a learning unit according to an experimental example of the present disclosure.

Referring to FIG. 18, for physicochemical profiling of macrophages according to the experimental example, the learning unit 50 may segment the acquired near-infrared image into a plurality of segmentation images, annotate each of the cells cl included in the segmentation image, and distinguish each of the cells cl with the bounding box bx.

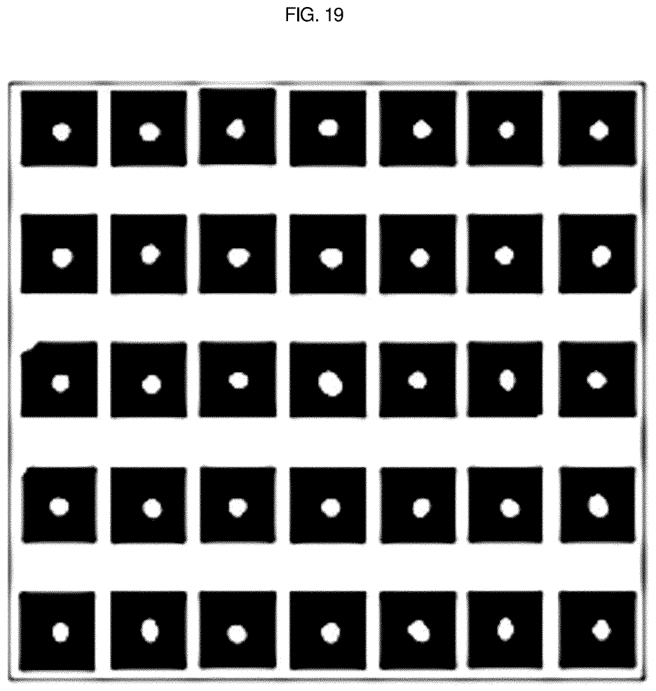

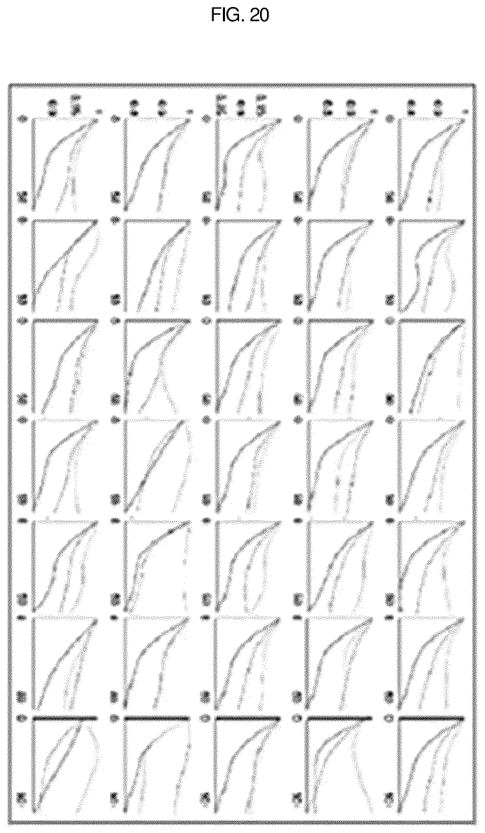

FIGS. 19 and 20 are views for describing an analysis method of an analysis unit according to the experimental example of the present disclosure.

Referring to FIGS. 19 and 20, the analysis unit 40 may extract the biophysical database managed by the learning unit 50 for the cell cl of the acquired image, or extract the biochemical database managed by the learning unit 50 for the cell cl of the acquired image. The biophysical database may include at least one of a size, a shape, and a refractive index of the cell cl, and the biochemical database may include a nitric oxide efflux rate of the cell cl.

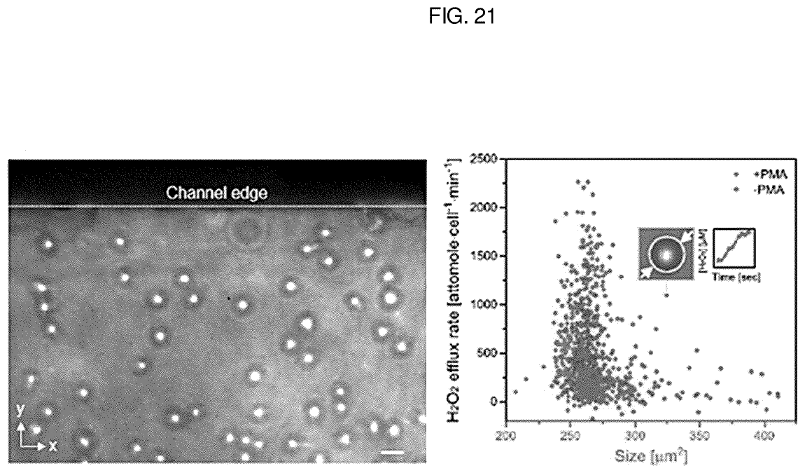

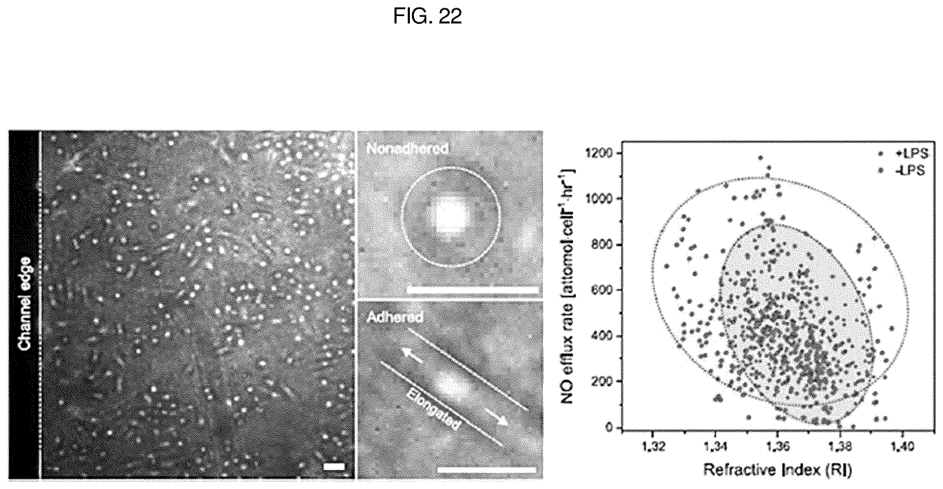

FIG. 21 is a view for describing immune heterogeneity of human monocyte reactive oxygen species (ROS), which is analyzed through a cell analysis system according to the experimental example of the present disclosure, and FIG. 22 is a view for describing heterogeneity of inducible nitric oxide synthase (iNOS) of a giant cell of a mouse, which is analyzed through the cell analysis system according to the experimental example of the present disclosure.

Referring to FIGS. 21 and 22, through the cell analysis system 100 according to the experimental example of the present disclosure, immune heterogeneity of human monocyte reactive oxygen species (ROS) and heterogeneity of inducible nitric oxide synthase (iNOS) a giant cell of a mouse may be analyzed.

Accordingly, it may be proven that the cell analysis system 100 may be applied to the analysis of the human monocyte reactive oxygen species (ROS) and the giant cell of the mouse.

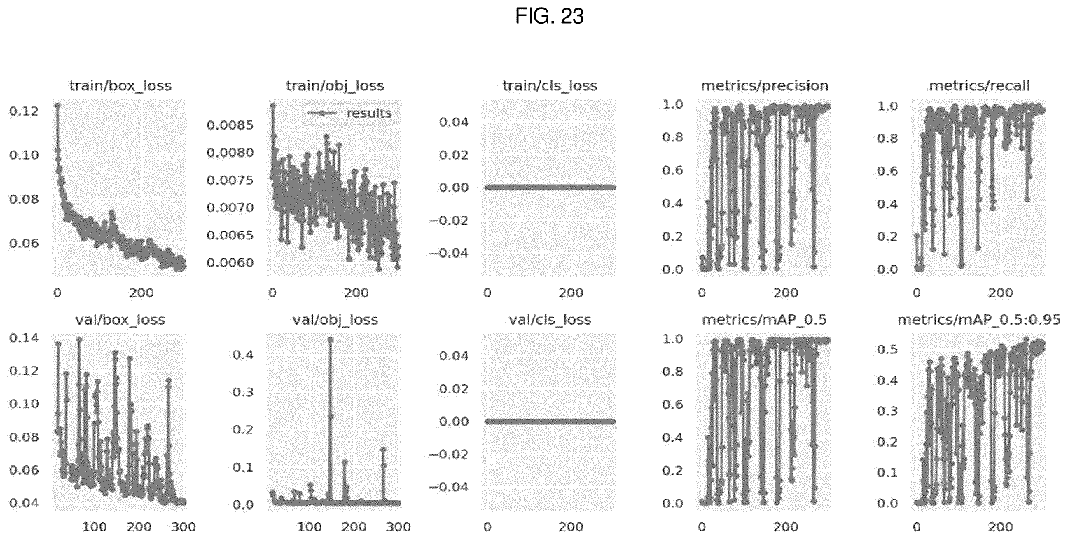

FIG. 23 is a view for describing the learning unit according to the experimental example of the present disclosure.

Referring to FIG. 23, loss indicators box_loss, obj_loss, and cls_loss and precision indicators precision, recall, and mAP may be observed during performance, training, and validation stages of a machine learning model of the learning unit 50. In particular, a degree of error that the learning model exhibits in positioning, object recognition, classification accuracy, and the like may be observed through the loss indicators box_loss, obj_loss, and cls_loss.

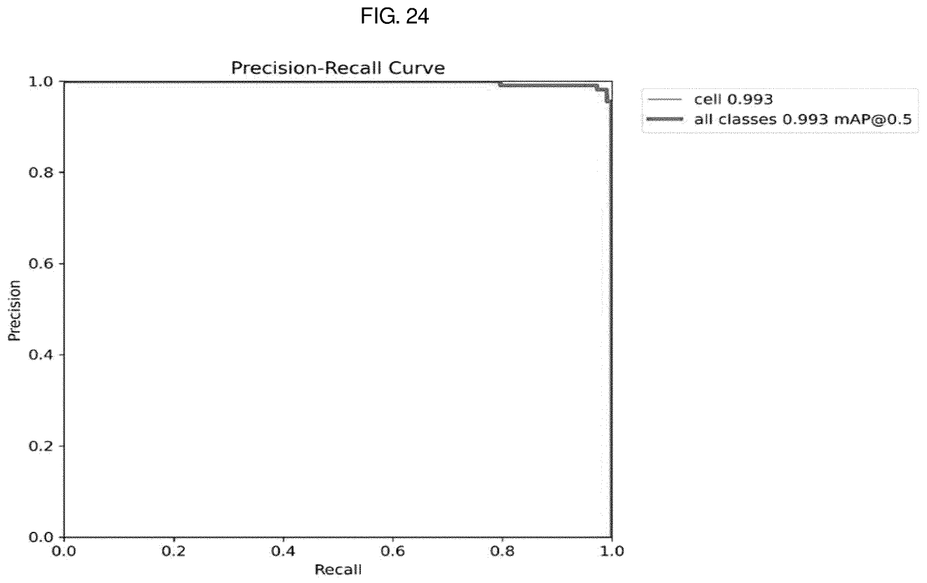

FIG. 24 is a view for describing a model according to the experimental example of the present disclosure.

Referring to FIG. 24, a proportion of positive samples that are correctly predicted by the model and a proportion of positive samples that are correctly identified by the model among all actual positive samples may be observed through a precision-recall curve.

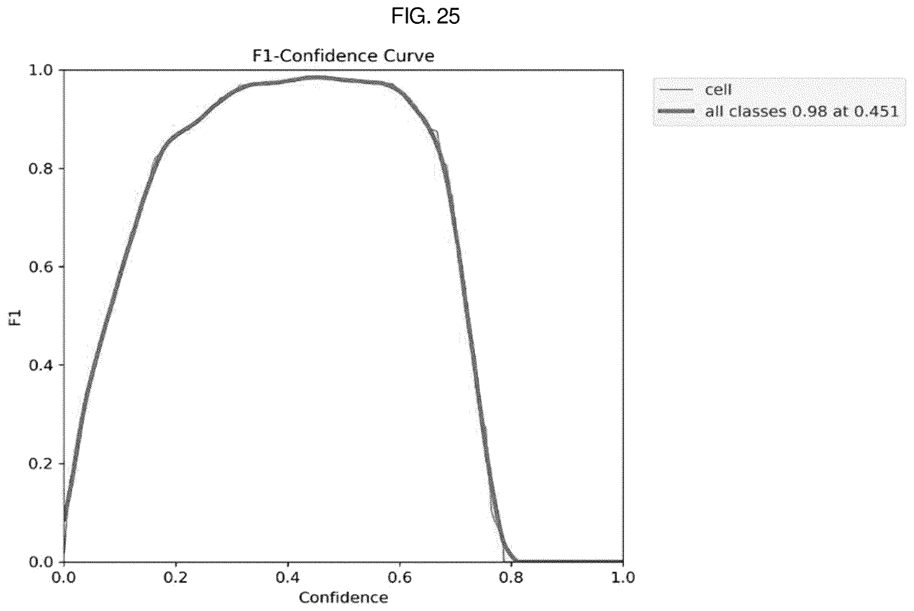

FIG. 25 is a view for describing a variation curve of an F1 score according to a confidence threshold according to the experimental example of the present disclosure.

Referring to FIG. 25, YOLOv5 may extract various properties by using various convolution kernels through labeled cell images, and a region that is not subjected to the imaging may be identified in the acquired image by using the properties. An output result of YOLOv5 may include a bounding box, classification, and confidence of an object. The bounding box may provide position and size information of the object within the image, and the classification may be used to predict a category to which the object belongs. The confidence may probabilistically represent accuracy of prediction, and, when the confidence is high, it may mean that accuracy of prediction of a model is excellent. According to the experimental example of the present disclosure, 0.451 was set as a threshold, only prediction with higher confidence than the threshold was considered as effective detection, and uncertain prediction was filtered to ensure accuracy and confidence of a final result. When YOLOv5 is used, prediction frames around respective cells may have different colors according to a type of the cell or a specific chemical material released from or absorbed by the cell. In addition, the color of the prediction frame may be changed from a deep color to a shallow color according to the confidence, so that a state of the cell cl may be recognized more rapidly and easily.

According to the experimental example of the present disclosure, the acquired image having a size of 640×512 was segmented into a size of 100×100, and images obtained through the segmenting overlapped by a size of 20 pixels, so that substantially all of the cells cl included in the image may be analyzed. Next, intensities of all the acquired images were set to be uniform, and the intensities of all the acquired images were averaged. Thereafter, a linear transformation scheme of Python was used to adjust an average intensity value of each of the images to an average intensity value of an entire image so that a detection effect may be improved. After the result of YOLOv5 is obtained as described above, an effect of training according to the experimental example of the present disclosure was evaluated by using precision and an F1 score.

Subsequently, the cells cl and a background were separated from each other in the image by using a binarization scheme, an average intensity was calculated to acquire an image having only the cells cl without the background, and an area was measured. In addition, a pixel position of the separated cell cl was stored, and an average intensity value was calculated. In order to obtain eccentricity of the cell cl, the longest axis, the shortest axis, and a center point of the cell cl were determined by using a five-point method, and ellipticity was calculated by using a least square method.

According to the experimental example of the present disclosure, as shown in FIG. 25, the F1 score for all categories reached 0.98 at a confidence threshold of 0.451, so that high precision and an excellent training result were proven.

FIG. 26 is a graph showing learning curves and losses thereof of the model according to the experimental example of the present disclosure.

Referring to FIG. 26, when the imaged cell cl is learned by applying the model by the learning unit 50 according to the experimental example of the present disclosure, the learning was repeatedly performed to reduce a loss.

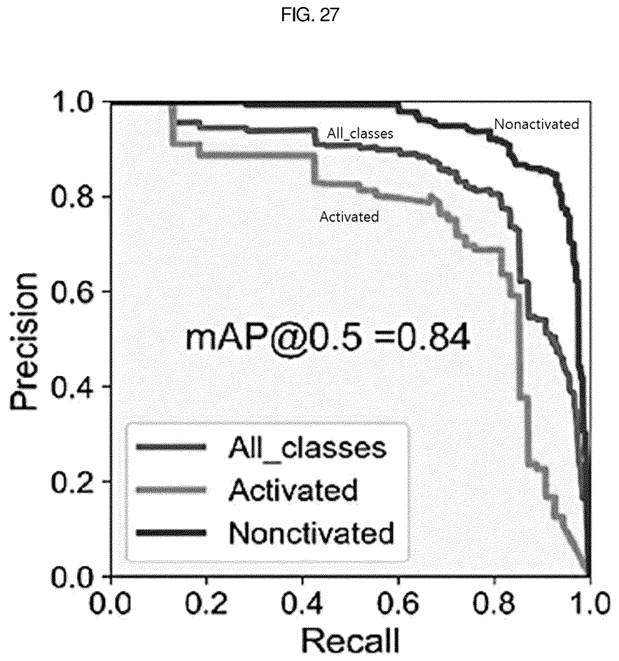

FIG. 27 is a graph showing relation between precision and recall of the model according to the experimental example of the present disclosure.

Referring to FIG. 27, an area under a curve in the graph may represent average precision (AP), and, as shown in FIG. FIG. 27, precision and recall of the model according to the experimental example of the present disclosure were excellent.

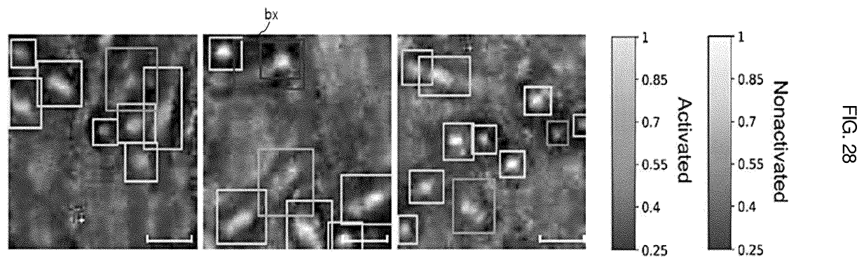

FIG. 28 is a view for describing an imaging result of a cell according to the experimental example of the present disclosure.

Referring to FIG. 28, in the near-infrared image according to the experimental example of the present disclosure, the imaging was performed well with the bounding box bx for each of the cells cl. In addition, in FIG. 28, a color of the bounding box bx may correspond to a confidence bar on a right side, and the confidence may be evaluated for each color of the bounding box bx.

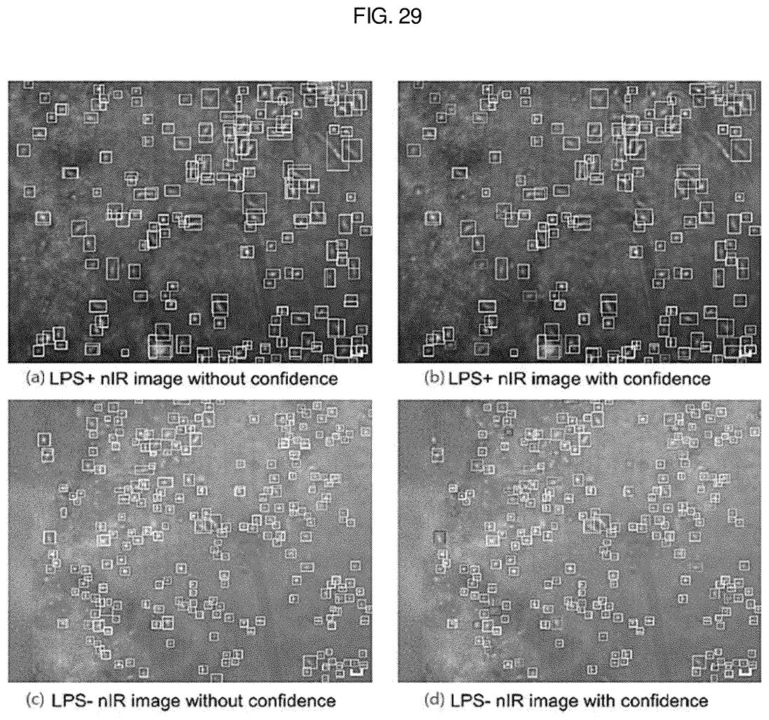

FIG. 29 shows an imaging result to which a result of performing learning by the learning unit is applied according to the experimental example of the present disclosure.

Referring to FIGS. 29(a) and 29(b), according to the experimental example of the present disclosure, results of the imaging when the macrophages are activated with lipopolysaccharides from Escherichia coli O55:B5 (LPS (Escherichia coli O55:B5)) may be observed, and, referring to FIGS. 29(c) and 29(d), results of the imaging when the macrophages are nonactivated with lipopolysaccharides from Escherichia coli O55:B5 (LPS (Escherichia coli O55:B5)) may be observed.

In addition, referring to FIGS. 29(b) and 29(d), according to the experimental example of the present disclosure, results of the imaging with confidence may be observed, and, referring to FIGS. 29(a) and 29(c), results of the imaging without confidence may be observed.

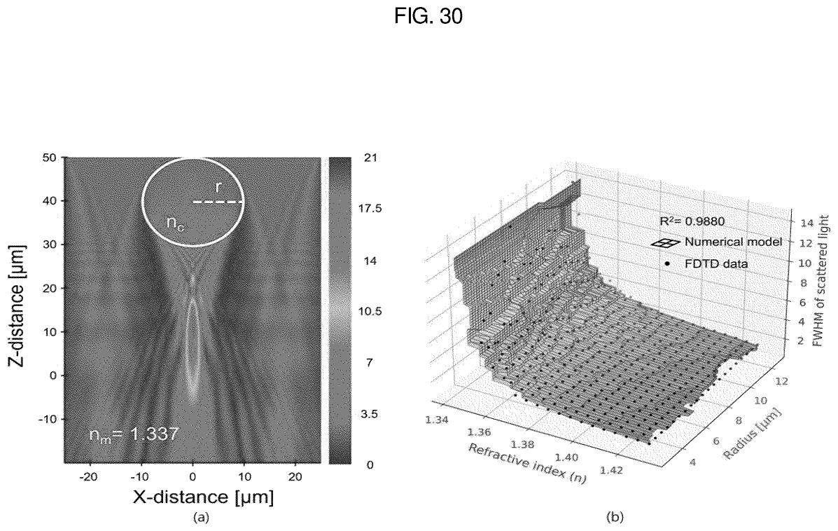

FIG. 30 is a view for describing relation among a size, a refractive index, and a full width at half maximum (FWHM) of the cell according to the experimental example of the present disclosure.

Referring to FIG. 30(a), according to the experimental example of the present disclosure, a nanoscale commercial electromagnetic wave simulation program (finite-difference time-domain, FDTD) was used to derive a refractive index and a size of the cell cl and recognize the photonic nanojet P generated by the cell cl.

Referring to FIG. 30(b), according to the experimental example of the present disclosure, the size and the refractive index of the cell cl may be substituted to derive a full width at half maximum (FWHM) at a position of the photonic nanojet P. According to the experimental example of the present disclosure, a relational formula among the size, the refractive index, and the full width at half maximum (FWHM) of the cell cl was established, and a numerical value of the refractive index was modeled, in which the size (radius) of the cell cl was set in a unit of 0.5 μm from 3 μm to 12.5 μm, and the refractive index was set in a unit of 0.005 from 1.345 to 1.43.

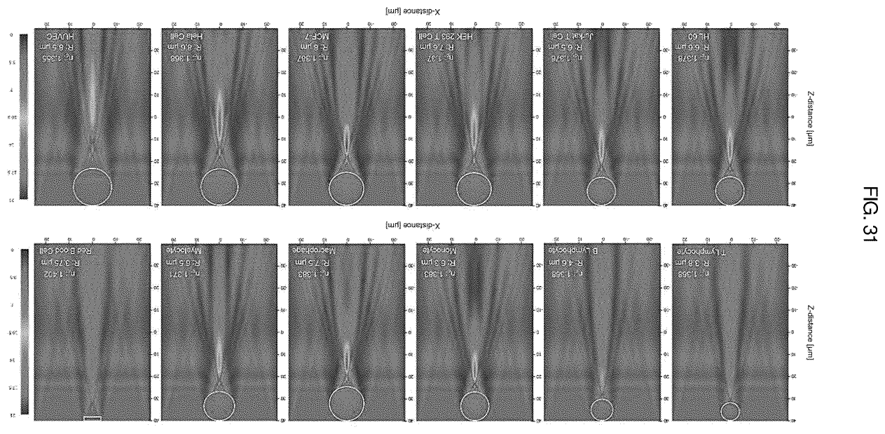

FIG. 31 is a view for describing sizes, refractive indexes, and photonic nanojets of various cells according to the experimental example of the present disclosure.

Referring to FIG. 31, according to the experimental example of the present disclosure, the nanoscale commercial electromagnetic wave simulation program (FDTD) was used to derive refractive indexes and sizes of various cells cl and recognize photonic nanojets P generated by the various cells cl.

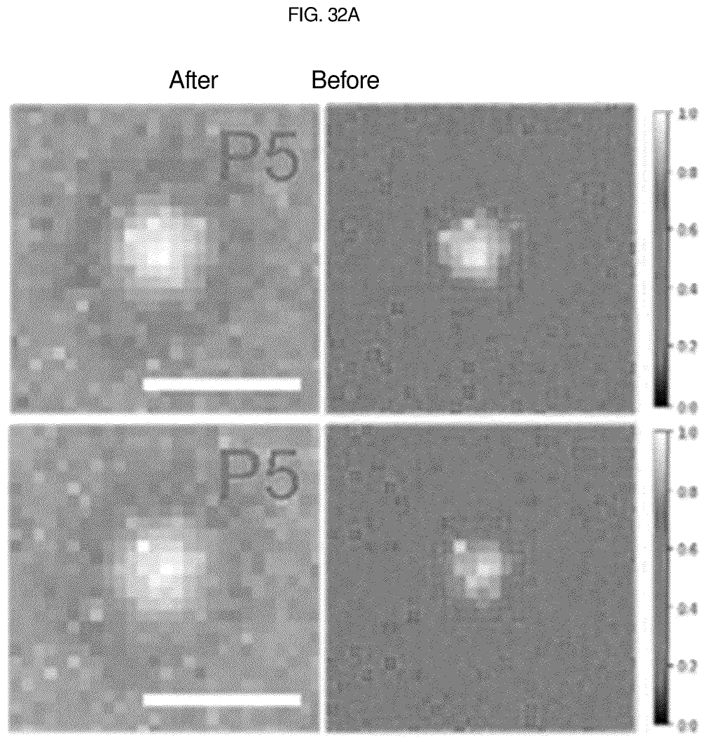

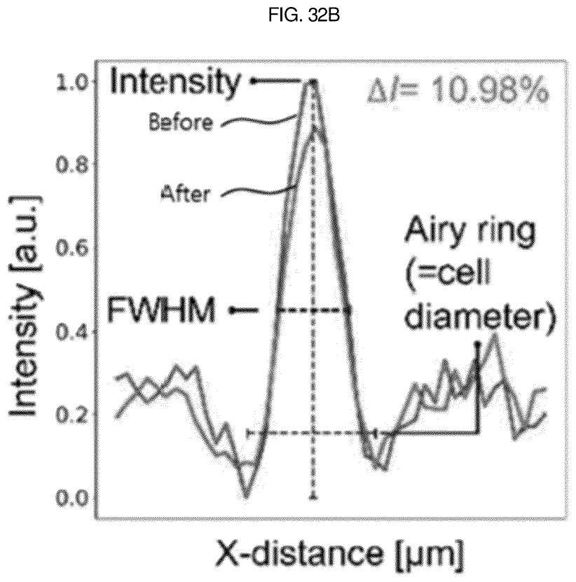

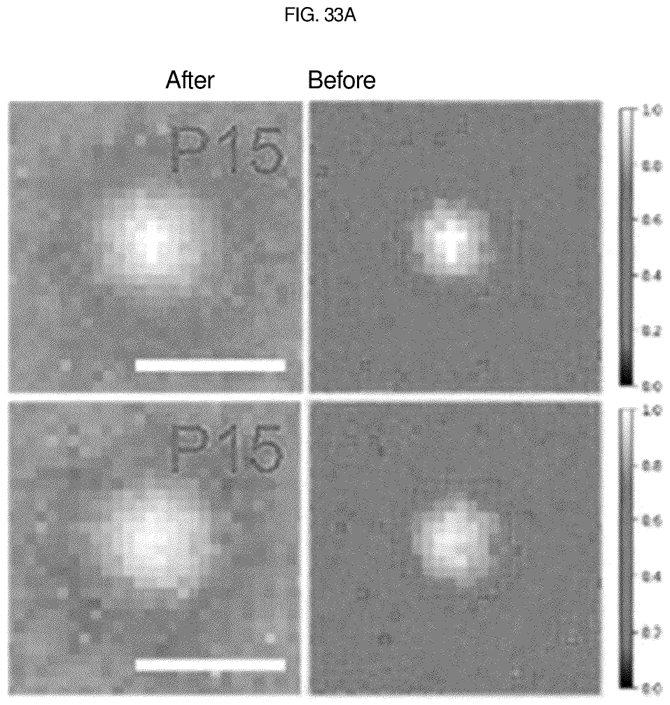

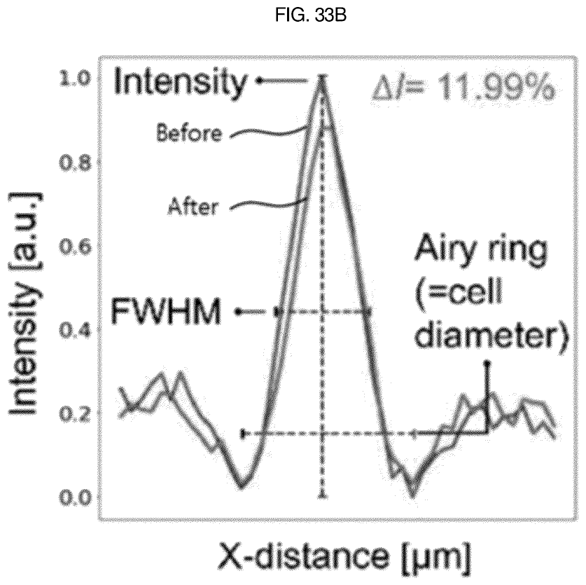

FIGS. 32A and 32B illustrate a near-infrared image and a full width at half maximum (FWHM) calculation result of a passage number 5 (P5) cell in a human dermal fibroblast (HDF) according to the experimental example of the present disclosure, and FIGS. 33A and 33B illustrate a near-infrared image and a full width at half maximum (FWHM) calculation result of a passage number 15 (P15) cell in a human dermal fibroblast (HDF) according to the experimental example of the present disclosure.

Referring to FIGS. 32 and 33, according to the experimental example of the present disclosure, an intensity variation graph may be derived from the near-infrared image, and the full width at half maximum (FWHM) may be calculated. In more detail, a size, eccentricity, a refractive index, and reactive oxygen species (ROS) of each of the P5 and P15 cells may be measured. First, in order to measure the reactive oxygen species (ROS) of the P5 and P15 cells, the near-infrared images were taken twice at a time interval of 10 minutes to check an intensity variation. Positions of the P5 and P15 cells were recognized in the near-infrared image through AI (YOLOv5), and the near-infrared image was segmented. In this case, for accurate measurement of the reactive oxygen species (ROS), a cell having a changed position in the image taken after 10 minutes was not processed. In this case, only a case where each coordinate differs within 1% was determined to match. The P5 and P15 cells were marked with ellipses in the segmented near-infrared image, and a size and eccentricity of the marked ellipse were determined. In addition, after the intensity variation graph is drawn on a straight line passing through a center of the ellipse, a full width at half maximum (FWHM) in the graph was calculated.

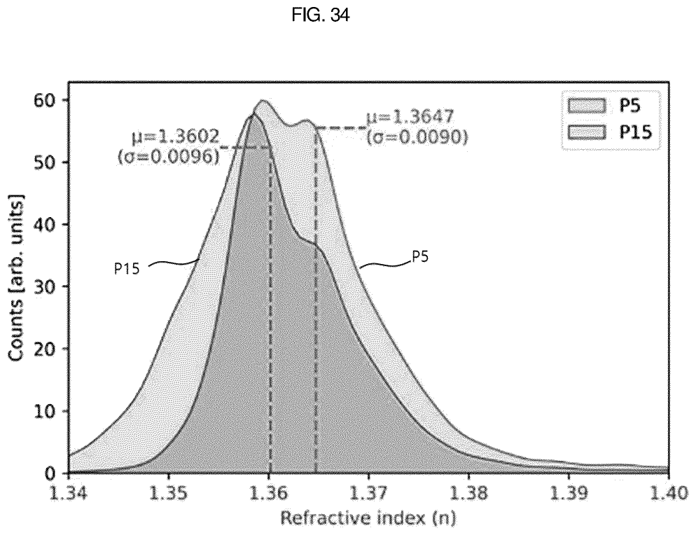

FIG. 34 is a graph showing refractive indexes of the P5 and P15 cells in the human dermal fibroblast (HDF) according to the experimental example of the present disclosure.

Referring to FIG. 34, according to the experimental example of the present disclosure, the nanoscale commercial electromagnetic wave simulation program (FDTD) was used, and the full width at half maximum (FWHM) calculated through FIG. 33 was used, so that the refractive indexes of the P5 and P15 cells may be derived. Accordingly, the reactive oxygen species (ROS) of the P5 and P15 cells may be derived.

In more detail, hydrogen peroxide (H₂O₂) may cause a chemical reaction with the light emitting layer 12, that is, the single-wall carbon nanotube (SWNT), to form an H₂O₂-SWNT complex. The reaction at this point may follow Formula 1 below.

$${H}_{2} {O}_{2} + S WNT ↔ {H}_{2} {O}_{2} - SWNT \qquad < Formula 1 >$$

An intensity variation rate of a wavelength measured by the flow unit 10 may vary according to a concentration of hydrogen peroxide (H₂O₂), K_{D} may represent a degree of separation of the H₂O₂-SWNT complex, and the intensity variation rate may be expressed as Formula 2 below.

$$\frac{I}{{I}_{o}} = \frac{{K}_{D}}{{K}_{D} + {[ {H}_{2} {O}_{2} ]}_{s e n s o r}} \qquad < Formula 2 >$$

A concentration variation rate of the H₂O₂-SWNT complex over time may be determined by a forward reaction rate constant K_{f} and a reverse reaction rate constant Kᵣ, and the concentration variation rate may be expressed as Formula 3 below.

$$< Formula 3 >$$

$$\frac{d [ {H}_{2} {O}_{2} - SWNT ]}{dt} = {k}_{f} [ {H}_{2} {O}_{2} ] [ SWNT ] - {k}_{r} [ {H}_{2} {O}_{2} - SWNT ]$$

In this case, K_{f} may be 8.68×10-4 (μMs)−1, and Kᵣ may be 3.18×10−3 s−1.

Based on the information described above, an intensity over time may be modeled as Formula 4 below.

$$I ( t ) = {I}_{o} / {K}_{s} ( {K}_{r} + {K}_{f} [ {H}_{2} {O}_{2} ] {e}^{- {K}_{s} t} ) \qquad < Formula 4 >$$

After a sufficient time has elapsed, K_{D} may be calculated to be 0.00204 M. Among the hydrogen peroxide (H₂O₂) released from the P5 and P15 cells, an amount of hydrogen peroxide (H₂O₂) making contact with the flow unit 10 may be 0.193 times according to Formula 5 below.

$$I ( t ) = {I}_{o} / {K}_{s} ( {K}_{r} + {K}_{f} [ {H}_{2} {O}_{2} ] {e}^{- {K}_{s} t} ) \qquad < Formula 5 >$$

The intensity variation rate of the wavelength measured by the flow unit 10, an amount of hydrogen peroxide (H₂O₂) making contact with the P5 and P15 cells, and the K_{D} value were substituted to obtain a value of hydrogen peroxide (H₂O₂) released from the P5 and P15 cells, and calculated volume values of the P5 and P15 cells were substituted to quantify an amount of hydrogen peroxide (H₂O₂) generated per cell of the P5 and P15 cells. In this case, an average intensity of a background except for a region of interest marked with an ellipse was adjusted to be the same in the near-infrared images taken at the time interval of 10 minutes, so that an influence of sensor quenching over time was minimized. The result values obtained as described above were used in analyzing the P5 and P15 cells.

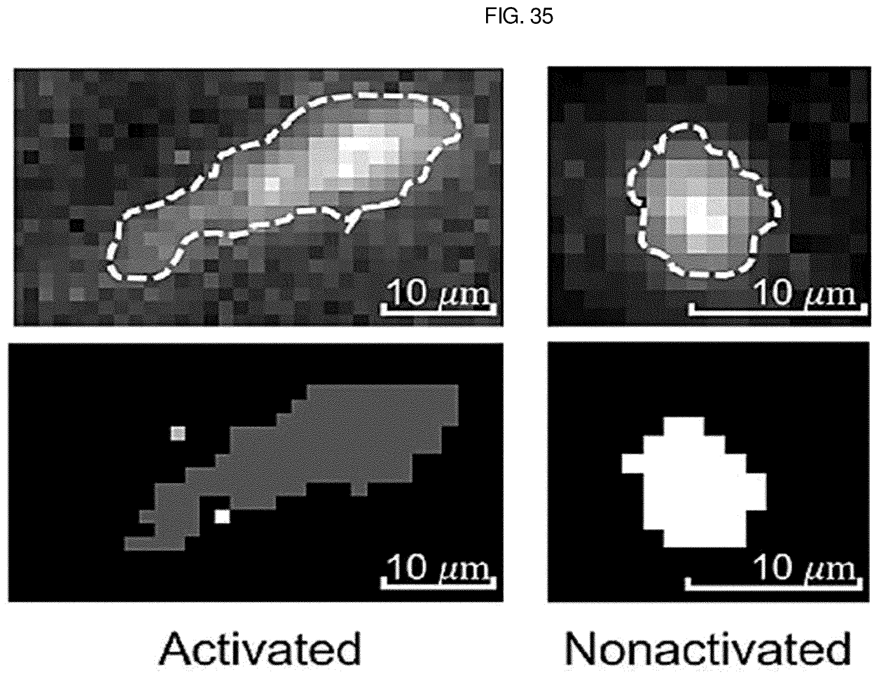

FIG. 35 is a view showing a state in which an activated macrophage (Activated) and a nonactivated macrophage (Nonactivated) are spatially separated from a background in a near-infrared image according to the experimental example of the present disclosure.

Referring to FIGS. 35, according to the experimental example of the present disclosure, first, the acquired image may be converted into a grayscale image. In more detail, the acquired image may be converted into the grayscale image after an average pixel value is calculated. In addition, the grayscale image may be adjusted through adaptive histogram equalization. In addition, a background brightness of the grayscale image may be adjusted to be uniform. For this reason, data acquired from the grayscale image may be uniform.

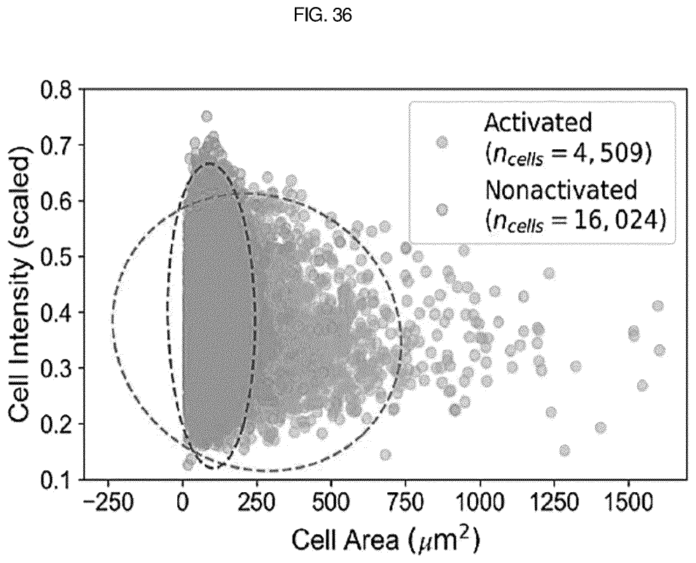

FIG. 36 is a graph showing relation between an intensity and an area of the activated macrophage (Activated) and the nonactivated macrophage (Nonactivated) according to the experimental example of the present disclosure.

Referring to FIG. 36, according to the experimental example of the present disclosure, a confidence circle may be expressed as σ=3, and the number of nonactivated macrophages (Nonactivated) was greater than the number of activated macrophages (Activated), while the activated macrophage (Activated) had a higher intensity and a wider area than the nonactivated macrophage (Nonactivated).

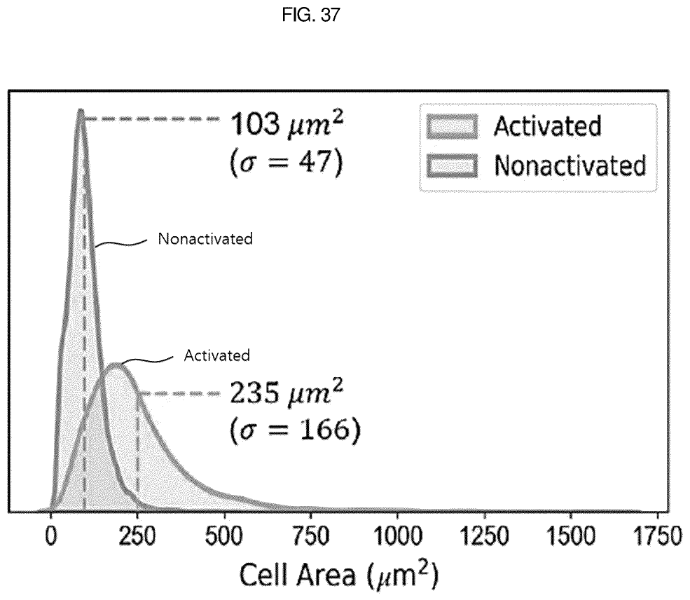

FIG. 37 is a graph showing area distribution of the activated macrophage (Activated) and the nonactivated macrophage (Nonactivated) according to the experimental example of the present disclosure.

Referring to FIG. 37, according to the experimental example of the present disclosure, area distribution of the activated macrophages (Activated) was wider than area distribution of the nonactivated macrophages (Nonactivated).

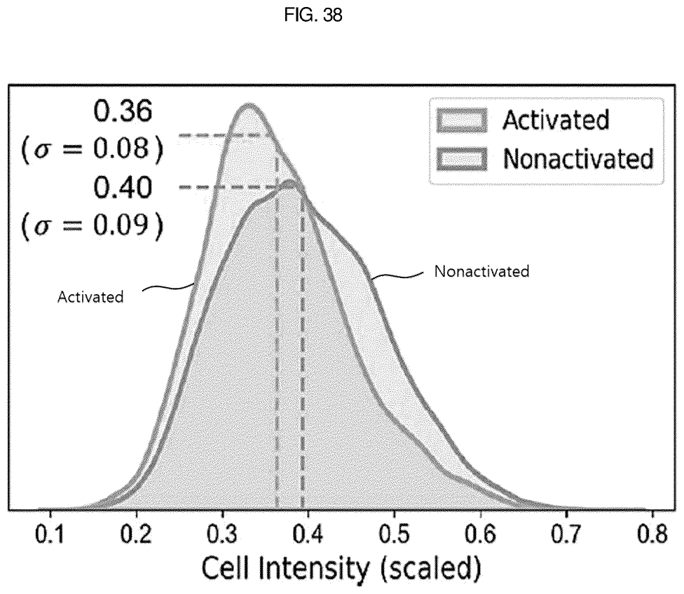

FIG. 38 is a graph showing intensity distribution of the activated macrophage (Activated) and the nonactivated macrophage (Nonactivated) according to the experimental example of the present disclosure.

Referring to FIG. 38, according to the experimental example of the present disclosure, intensity distribution of the activated macrophages (Activated) was wider than intensity distribution of the nonactivated macrophages (Nonactivated).

Although the exemplary embodiments of the present disclosure have been described in detail above, the scope of the present disclosure is not limited to a specific embodiment, and shall be interpreted by the appended claims. In addition, it is to be understood by a person having ordinary skill in the art that various changes and modifications can be made without departing from the scope of the present disclosure.

What is claimed is:

1. A cell analysis method comprising:
providing a flow unit configured to provide a path in which a cell flows, the flow unit including a light emitting layer configured to, in response to being irradiated by a first light in a first wavelength band, emit a second light in a second wavelength band;
irradiating the light emitting layer with the first light;
emitting the second light from the light emitting layer in response to the irradiation of the first light;
acquiring an image formed by reacting the cell with the second light emitted from the light emitting layer; and
analyzing the cell from the acquired image.

2. The cell analysis method of claim 1, further comprising:
learning the cell,
wherein the learning includes:
converting the acquired image into a grayscale image;
segmenting the grayscale image into a plurality of segmentation images;
imaging the cell in the segmentation image;
learning the imaged cell by applying a model; and
managing a result related to the learned cell in a database.

3. The cell analysis method of claim 2, wherein the segmenting includes segmenting at least a portion overlaps in the segmentation images obtained through the segmenting.

4. The cell analysis method of claim 2, wherein the managing includes:
managing a biophysical result of the learned cell in a biophysical database; or
managing a biochemical result of the learned cell in a biochemical database.

5. The cell analysis method of claim 4, wherein the analyzing further includes at least one of:
extracting the biophysical database for the cell from the acquired image;
extracting the biochemical database for the cell from the acquired image; or
a combination thereof.

6. The cell analysis method of claim 5, wherein the biophysical database includes at least one of a size, a shape, a refractive index of the cell, or any combination thereof, and
wherein the biochemical database includes a nitric oxide efflux rate of the cell.

7. The cell analysis method of claim 1, wherein a number of cells flowing in the flow unit is between 10⁵ and 10⁷ per minute.

8. The cell analysis method of claim 1, wherein the first light includes a visible light, and
wherein the second light includes a near-infrared light.

9. A cell analysis system comprising:
a flow unit configured to provide a path in which a cell flows, the flow unit including a light emitting layer configured to, in response to being irradiated by a first light in a first wavelength band, emit a second light in a second wavelength band;
an acquisition unit configured to acquire an image formed by reacting the cell with the second light; and
an analysis unit configured to analyze the cell from the image acquired by the acquisition unit.

10. The cell analysis system of claim 9, further comprising:
an irradiation unit configured to irradiate the light emitting layer with the first light, wherein the irradiation unit includes:
a laser configured to perform the irradiation of the first light;
a diffuser lens configured to diffuse the first light subjected to the irradiation by the laser; and
a convex lens configured to collect the first light diffused by the diffuser lens.

11. The cell analysis system of claim 10, wherein the acquisition unit includes:
a stage on which the flow unit is seated, the stage including a transmission layer configured to transmit the first light through the seated flow unit;
a beam splitter configured to reflect the first light subjected to the irradiation by the irradiation unit toward the flow unit seated on the stage;
an objective lens configured to collect the first light reflected by the beam splitter to provide the collected first light to the light emitting layer on the stage; and
a camera configured to form an image from a reaction signal of the cell with the second light emitted from the light emitting layer in response to the irradiation of the first light.

12. The cell analysis system of claim 10, wherein the acquisition unit and the convex lens have at least one of:
a first separation distance of between 8 cm and 12 cm;
a second separation distance of between 30.5 cm and 34.5 cm; or
a third separation distance of between 39 cm and 43 cm.

13. The cell analysis system of claim 12, wherein the laser is configured to perform the irradiation of the first light having a power of between 100 mW and 500 mW in a wavelength band of between 560 nm and 750 nm,
wherein the convex lens has a diameter of between 30 mm and 70 mm, and a focal length of between 80 mm and 120 mm, and
wherein the diffuser lens has a diameter of between 5 mm and 45 mm, and a grit of between 100 mm and 140 mm.

14. The cell analysis system of claim 12, wherein, for the first to third separation distances, a magnification power of the objective lens is between a magnification power of 5 and 20.

15. The cell analysis system of claim 12, wherein, for the first to third separation distances, a number of cells flowing in the flow unit is between 10⁵ and 10⁷ per minute.

16. The cell analysis system of claim 9, further comprising:
a learning unit configured to learn the cell,
wherein the learning unit is configured to:
learn the cell by segmenting the acquired image into a plurality of segmentation images, such that at least a portion overlaps in the segmentation images obtained through the segmenting; and
manage a result related to the learned cell in a database.

17. The cell analysis system of claim 16, wherein the learning unit is further configured to:
manage a biophysical result of the learned cell in a biophysical database; or
manage a biochemical result of the learned cell in a biochemical database.

18. The cell analysis system of claim 17, wherein the analysis unit is further configured to:
extract the biophysical database managed by the learning unit for the cell of the acquired image; or
extract the biochemical database managed by the learning unit for the cell of the acquired image.

19. The cell analysis system of claim 18, wherein the biophysical database includes at least one of a size, a shape, a refractive index of the cell, or any combination thereof, and
wherein the biochemical database includes a nitric oxide efflux rate of the cell.

20. The cell analysis system of claim 9, wherein the first light includes a visible light, and
wherein the second light includes a near-infrared light.
